(19) United States
(12) Patent Application Publication
(10) Pub. No.: US 2023/0143932 A1
(43) Pub. Date: May 11, 2023

(54) PIN6 PROTEINS FOR THE FORMATION OF NODULE-LIKE STRUCTURES

(71) Applicant: University of Freiburg, Freiburg (DK)

(72) Inventors: Franck Anicet Ditengou, Gundelfingen (DE); Thomas Ott, Merzhausen (DE)

(21) Appl. No.: 17/933,280

(22) Filed: Sep. 19, 2022

Related U.S. Application Data
(60) Provisional application No. 63/246,031, filed on Sep. 20, 2021.

(51) Int. Cl. C12N 15/82; C12Q 1/6895; G01N 33/74

(52) U.S. Cl. CPC C12N 15/8273; C12N 15/8203; C12Q 1/6895; G01N 33/74; C12Q 2600/13; C12Q 2600/158; C12N 2740/00043

(57) ABSTRACT

The present disclosure provides DNA molecules and constructs, including their nucleotide sequences, useful for expressing proteins in plants to promote formation of nodule-like structures in the presence of Rhizobia. The present disclosure also provides DNA molecules and constructs, including their nucleotide sequences, useful for expressing proteins in plants to increase drought resistance. The present disclosure also provides plants and plant cells transgenic plants, plant cells, plant parts, seeds, and commodity products comprising the DNA molecules, along with methods of their use.

### CROSS REFERENCE TO RELATED APPLICATIONS

This application claims the benefit of U.S. Provisional Application No. 63/246,031, filed on Sep. 20, 2021, the entire content of which is hereby incorporated herein by reference.

### INCORPORATION OF SEQUENCE LISTING

The sequence listing that is contained in the file named “AGOE005US.xml”, which is 34.6 KB (as measured in Microsoft Windows®) and was created on Sep. 17, 2022, is filed herewith by electronic submission and is incorporated by reference herein.

### FIELD

The present disclosure relates to the field of plant molecular biology and plant genetic engineering, proteins useful for improving agronomic performance, and DNA molecules useful for modulating gene expression of the same in plants.

### BACKGROUND

Many of the world's farmers face pressure from nitrogen-deficient soils or drought conditions, each of which can result in low yield or plant death. Symbiotic bacteria can improve plant biomass under low-nitrogen conditions. For example, symbiosis with nitrogen-fixing bacteria allows fabaceae plants to exploit dinitrogen reduced by rhizobia in exchange for photosynthesis-derived carbon compounds from the host plant. The formation of root nodules is a hallmark of this symbiotic relationship. However, the formation of root nodules and the colonization of host cells by symbionts is an evolutionary achievement primarily related to fabacean plants. Thus, most plants outside of the fabacea family do not have access to dinitrogen reduced by rhizobia into ammonia, which is suitable for utilization by plants. Therefore, methods for overexpressing proteins involved in the formation of nodule-like structures required for symbiotic nitrogen fixation in plants are needed to provide farmers with crop plants exhibiting improved agronomic performance.

### SUMMARY

The instant disclosure provides DNA molecules and constructs, including their nucleotide sequences, useful for expressing proteins in plants to promote the formation of nodule-like structures; and increase drought resistance. The proteins as disclosed herein can be used alone or in combination with other proteins in planta, thus providing alternative means to form nodule-like structures required for symbiotic nitrogen fixation; and increase drought resistance. The present disclosure also provides novel DNA molecules and constructs, including their nucleotide sequences, useful for modulating gene expression in plants and plant cells. Furthermore, the disclosure also provides transgenic plants, plant cells, plant parts, seeds, and commodity products comprising the DNA molecules as described herein, along with methods of their use. In one embodiment, disclosed in this application is a recombinant DNA molecule comprising a heterologous promoter operably linked to a polynucleotide segment encoding an auxin transporter protein or fragment thereof, wherein the auxin transporter protein comprises the amino acid sequence of any of SEQ ID NOs: 3 or 5-14; or the auxin transporter protein comprises an amino acid sequence having at least 85%, or 90%, or 95%, or 98% or 99%, or about 100% amino acid sequence identity to any of SEQ ID NOs: 3 or 5-14; or the polynucleotide segment hybridizes under stringent hybridization conditions to a polynucleotide having the nucleotide sequence of SEQ ID NO: 1, 2, or 4; or the polynucleotide segment comprises the nucleotide sequence of SEQ ID NO: 1, 2, or 4. The recombinant DNA molecule can comprise a sequence that functions to express the auxin transporter protein in a plant, and which when expressed in a plant produces the formation of nodule-like structures in the presence of rhizobia; or increases drought resistance as compared to a plant lacking said recombinant nucleic acid molecule. In another embodiment of this application, the recombinant DNA molecule is present within a bacterial or plant host cell. Contemplated bacterial host cells include at least the genus of Agrobacterium, Rhizobium, Bacillus, Brevibacillus, Escherichia, Pseudomonas, Klebsiella, Pantoea, and Erwinia. In certain embodiments, the Bacillus species is a Bacillus cereus or Bacillus thuringiensis, the Brevibacillus is a Brevibacillus laterosporus, or the Escherichia is an Escherichia coli. Contemplated plant host cells include a dicotyledonous plant cell and a monocotyledonous plant cell. Contemplated plant cells further include alfalfa, banana, barley, bean, broccoli, cabbage, brassica, carrot, cassava, castor, cauliflower, celery, chickpea, Chinese cabbage, citrus, coconut, coffee, corn, clover, cotton (Gossypium sp.), a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell. In another embodiment, the auxin transporter protein exhibits activity in the presence of bacteria or fungi, including but not limited to Mesorhizobium loti, Sinorhizobium meliloti, Sinorhizobium fredii, Rhizobium sp. IRBG74 and NGR234, Bradyrhizobium sp., and Azorhizobium caulinodans ORS571. In another embodiment, the auxin transporter protein or functional fragment thereof exhibits root nodulation activity in the presence of bacteria or fungi, including but not limited to Mesorhizobium loti, Sinorhizobium meliloti, Sinorhizobium fredii, Rhizobium sp. IRBG74 and NGR234, Bradyrhizobium sp., and Azorhizobium caulinodans ORS571. Also contemplated in this application are bacteria and plants and plant parts comprising a recombinant DNA molecule encoding an auxin transporter protein or fragment thereof. The recombinant molecule (e.g. construct) may comprise a heterologous promoter for expression in bacterial or plant cells of the operably linked polynucleotide segment encoding the auxin transporter protein. In certain embodiments, a plant or plant part comprising a recombinant DNA molecule encoding an auxin transporter protein or fragment thereof may be a non-legume plant. In some embodiments, a plant or plant part comprising a recombinant DNA molecule encoding an auxin transporter protein or fragment thereof may be a non-host plant, wherein said plant does not normally form a symbiotic relationship with nitrogen-fixing rhizobia. Both dicotyledonous plants and monocotyledonous plants are contemplated. In another embodiment, the plant is further selected from the group consisting of alfalfa, banana, barley, bean, broccoli, cabbage, brassica (e.g. canola), carrot, cassava, castor, cauliflower, celery, chickpea, Chinese cabbage, citrus, coconut, coffee, corn, clover, cotton (i.e. Gossypium sp.), a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn (i.e. maize) such as sweet corn or field corn, sweet gum, sweet potato, switchgrass, tea, tobacco, tomato, triticale, turf grass, watermelon, and wheat plant. The plant parts may for instance include, without limitation, leaves, tubers, roots, stems, seeds, embryos, flowers, inflorescences, bolls, pollen, fruit, animal feed, and biomass. Processed plant parts, for instance, wood, or oil, non-viable ground seeds or fractionated seeds, flour, or starch produced from the plant leaves, flowers, roots, seeds or tubers are also contemplated. Still further provided is a transgenic seed comprising the recombinant DNA molecules according to the instant disclosure. In still another embodiment, a composition comprising the recombinant nucleic acid molecules as disclosed herein is contemplated. The composition can further comprise a Rhizobium bacterium. In certain embodiments, the at least one Rhizobium bacterium is selected from the group consisting of Mesorhizobium loti, Sinorhizobium meliloti, Sinorhizobium fredii, Rhizobium sp. IRBG74 and NGR234, Bradyrhizobium sp., and Azorhizobium caulinodans ORS571. It is also contemplated that in certain embodiments, the composition can comprise a plant, plant cell, plant part, or seed comprising the recombinant nucleic acid molecules as disclosed herein. Commodity products comprising a detectable amount of the recombinant DNA molecules and disclosed proteins disclosed in this application are also contemplated. Such commodity products include commodity corn bagged by a grain handler, corn flakes, corn cakes, corn flour, corn meal, corn syrup, corn oil, corn silage, corn starch, corn cereal, and the like, and corresponding soybean, rice, wheat, sorghum, pigeon pea, peanut, fruit, melon, and vegetable commodity products including, where applicable, juices, concentrates, jams, jellies, marmalades, and other edible forms of such commodity products containing a detectable amount of such polynucleotides and or polypeptides of this application, whole or processed cotton seed, cotton oil, lint, seeds and plant parts processed for feed or food, fiber, paper, biomasses, and fuel products such as fuel derived from cotton oil or pellets derived from cotton gin waste, whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts. Also contemplated in this application is a method of producing seed comprising recombinant DNA molecules and the disclosed proteins. The method comprises planting at least one seed comprising the recombinant DNA molecules disclosed in this application; growing a plant from the seed; and harvesting seed from the plant, wherein the harvested seed comprises the referenced recombinant DNA molecules. Also disclosed in this application are methods for forming nodule-like structures in a plant, particularly a crop plant. The method comprises, in one embodiment, first expressing an auxin transporter protein or fragment thereof as set forth in any of SEQ ID NOs: 3 and 5-14 in a plant; or, alternatively, expressing an auxin transporter protein comprising an amino acid sequence having at least 85%, or 90%, or 95%, or 98% or 99%, or about 100% amino acid sequence identity to any of SEQ ID NOs: 3 or 5-14; and contacting said plant with an effective amount of one or more rhizobia bacterium. Nodule-like structures in plant or plant parts produced by the disclosed methods may express the auxin-like transporter protein disclosed herein. In certain embodiments, the method further comprises selecting a plant comprising nodule-like structures. Further provided herein is a method of detecting the presence of a recombinant nucleic acid molecule comprising a polynucleotide segment encoding an auxin transporter protein or fragment thereof, wherein: (a) said protein comprises the amino acid sequence of any of SEQ ID NOs: 3 or 5-14; (b) said protein comprises an amino acid sequence having at least 85%, or 90%, or 95%, or 98% or 99%, or about 100% amino acid sequence identity to any of SEQ ID NOs: 3 or 5-14; or (c) said polynucleotide segment hybridizes to a polynucleotide having the nucleotide sequence of SEQ ID NO: 1, 2, or 4. In one embodiment of the instant disclosure, the method comprises contacting a sample of nucleic acids with a nucleic acid probe that hybridizes under stringent hybridization conditions with genomic DNA from a plant comprising a polynucleotide segment encoding a protein or fragment thereof provided herein, and does not hybridize under such hybridization conditions with genomic DNA from an otherwise isogenic plant that does not comprise the segment, wherein the probe is homologous or complementary to SEQ ID NO: 1, 2, or 4, or a sequence that encodes a protein comprising an amino acid sequence having at least 85%, or 90%, or 95%, or 98% or 99%, or about 100% amino acid sequence identity to any of SEQ ID NOs: 3 or 5-14. The method may further comprise (a); subjecting the sample and probe to stringent hybridization conditions; and (b) detecting hybridization of the probe with DNA of the sample. Also provided herein by the instant disclosure are methods of detecting the presence of a protein or fragment thereof in a sample comprising protein, wherein said protein comprises the amino acid sequence of any of SEQ ID NOs: 3 or 5-14; or said protein comprises an amino acid sequence having at least 85%, or 90%, or 95%, or 98% or 99%, or about 100% amino acid sequence identity to any of SEQ ID NOs: 3 or 5-14. In one embodiment, the methods of detecting the presence of the protein or fragment thereof comprise: (a) contacting a sample with an immunoreactive antibody; and (b) detecting the binding of the antibody to the protein, thus confirming the presence of the protein in the sample. In some embodiments, the step of detecting comprises an ELISA, or a Western blot. Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

FIG. 1A-1G shows root organogenesis responses to transient auxin (2,4-Dichlorophenoxyacetic acid; 2,4-D) and cytokinin (6-Benzylaminopurine; BAP) treatments in Medicago truncatula (FIG. 1A), Lotus japonicus (FIG. 1B), and Hordeum vulgare (FIG. 1C). The average number of pseudonodules per plant (+/−standard errors) is given for n>30. Images of pseudonodules on Hordeum vulgare following synthetic auxin (2,4-Dichlorophenoxyacetic acid) treatment are shown (FIG. 1D-FIG. 1G).

FIGS. 2A-2B show over-expression of the auxin efflux carrier PIN6 in Arabidopsis impairs lateral root growth. An auxin signal (green) is visible in parental cells overlaying the growing lateral root until it emerges in WT roots (FIG. 2A). An auxin signal is absent in lateral roots overlaying tissues in plants overexpressing PIN6 (FIG. 2B). Ep, Co, and En in FIGS. 2A and 2B identify the epidermis, cortex, and endodermis, respectively.

FIGS. 3A-3B shows formation of nodule-like structures on roots of Arabidopsis PIN6 over-expressing lines in the presence of Rhizobia (IRBG74).

FIG. 4 shows images of PIN6/IRBG74-dependent nodule-like structures.

FIG. 5 shows fluorescent labeling, indicative of pericycle divisions, in and around PIN6/IRBG74-dependent nodule-like structures. Rhizobia IRBG74 express DsRed fluorescent tag (red).

FIG. 6 shows PIN6 overexpression in Arabidopsis can lead to increased drought resistance. Arabidopsis wild type plants (Col-0), pin6-knock-out mutants (pin6-5, pin6-6) and 35S::PIN6 (PIN6ox) grown in soil for 2 weeks and then confronted to drought by non-watering the plants for 15 days as shown.

FIGS. 7A-7D shows observed phenotype of barley lines expressing PIN6 under the control of an inducible promoter (ProZmOptXVE::PIN6). Plants were grown on sand and vermiculite (1:1) and were either induced (I) with ß-estradiol (dissolved in autoclaved tap water) or non-induced (NI) using water. Plants inoculated with Rhizobium sp. IRBG76-DsRed or Mesorhizobium loti-mCherry (OD: 0.001), respectively (FIG. 7A). PIN6 was induced in plants numbered 8-9 and 10-11 using ß-estradiol (FIG. 7B). Quantification of the number of stems per plant in induced and non-induced plants (FIG. 7C). Quantification of the height of the stalks in induced and non-induced plants (FIG. 7D).

FIG. 8 shows PIN6-expressing barley lines exhibiting nodule-like structures in the presence of IRBG74. Left (Panels A-D), T0 ProZmOptXVE:PIN6 (plant 6) non-induced (NI) with ß-estradiol (ß-Est) and inoculated with IRBG74 DsRed. Scale bars=2 mm. Right (Panels E-I), T0 ProZmOptXVE:PIN6 (plant 6) induced with ß-estradiol and inoculated with IRBG74 DsRed. White arrows indicate nodule-like structures along the root. Enlarged views of representative nodule-like structures (Panels F-I). Scale bars=500 μm (E), 20 μm (Panels F-I).

FIG. 9 shows PIN6 overexpression in Barley can lead to increased drought resistance. Barley plants (ProZmOptXVE::PIN6) grown on sand and vermiculite (1:1) were transferred to 50 ml Falcon tubes containing autoclaved tap water (Control), or water supplemented with 1 μM ß-estradiol (ß-EST), and left to grow for 2 weeks. ß-EST induced plants remained green whereas control plants were visibly desiccated.

FIG. 10A-10D depicts a sequence alignment of the Arabidopsis thaliana PIN1 protein At1g73590.1 (SEQ ID NO: 15), the Arabidopsis thaliana PIN2 protein At5g57090.1 (SEQ ID NO: 16), the Arabidopsis thaliana PIN3 protein At1g70940.1 (SEQ ID NO: 17), the Arabidopsis thaliana PIN4 protein At2g01420.1 (SEQ ID NO: 18), the Arabidopsis thaliana PIN5 protein At5g16530.1 (SEQ ID NO: 19), the Arabidopsis thaliana PIN6 protein At1g77110.1 (SEQ ID NO: 3), the Arabidopsis thaliana PIN7 protein At1g23080.1 (SEQ ID NO: 20), and the Arabidopsis thaliana PIN8 protein At5g15100.1 (SEQ ID NO: 21). The alignment was constructed using the Arabidopsis thaliana PIN6 protein At1g77110.1 (SEQ ID NO: 3) as the reference sequence identities normalized by alignment length. Colored by identity. Alignment based on Clustal omega (https://www.ebi.ac.uk)

FIG. 11A-11F depicts a sequence alignment of PIN6 proteins from Brachypodium distachyon (labeled “Bradi2g48170.1”; SEQ ID NO: 6), Musa acuminata (labeled “GSMUA_Achr3T22010”; SEQ ID NO: 7), Musa acuminata (labeled “GSMUA_Achr8T08300”; SEQ ID NO: 8), Arabidopsis thaliana (labeled “At1g77110.1”; SEQ ID NO: 3), Vitis vinifera (labeled “GSVIVG01010025001”; SEQ ID NO: 9), Solanum lycopersicum (labeled “Solyc06g059730”; SEQ ID NO: 10), Populus trichocarpa (labeled “Potri.005G187500.1”; SEQ ID NO: 11), Populus trichocarpa (labeled “Potri.002G072200.1”; SEQ ID NO: 12), Oryza sativa (labeled “LOC_Os01g51780.1”; SEQ ID NO: 13), Zea mays (labeled “GRMZM5G839411.02”; SEQ ID NO: 14). The alignment was constructed using the Arabidopsis thaliana PIN6 protein At1g77110.1 (SEQ ID NO: 3) as the reference sequence. Identities normalized by alignment length. Colored by identity. Alignment based on MUSCLE_v3.6. (http://aramemnon.uni-koeln.de).

FIG. 12: (a) shows transformed plants grown in soil+vermiculite. (b) shows nodule quantification demonstrating that PIN6-transformed plants form nodule-like structures but not control plants. (c) shows a root segment of PIN6-transformed plant inoculated with IRBG74 DsRed. Numbers indicate boxed areas showing nodule-like structures. (d) left panel shows bright field images of PIN6-transformed roots inoculated with IRBG74 DsRed, whereas the right panel shows PIN6-GFP and IRBG74 DsRed in these roots. The white arrow indicates a nodule structure. Scale bar, 50 μm.

### BRIEF DESCRIPTION OF THE SEQUENCES

SEQ ID NO:1 is a nucleic acid genomic sequence encoding the Arabidopsis thaliana PIN6 protein.

SEQ ID NO:2 is a nucleic acid sequence encoding the Arabidopsis thaliana PIN6 protein.

SEQ ID NO: 3 is the amino acid sequence of the Arabidopsis thaliana PIN6 protein, encoded by SEQ ID NO: 2.

SEQ ID NO: 4 is a nucleic acid sequence encoding the Lotus japonicus PIN6 protein.

SEQ ID NO: 5 is an amino acid sequence comprising the Lotus Japonicus PIN6 protein.

SEQ ID NO: 6 is the amino acid sequence of the Brachypodium distachyon PIN6 protein. Bradi2g48170.1.

SEQ ID NO: 7 is the amino acid sequence of the Musa acuminate PIN6 protein GSMUA_Achr3T22010.

SEQ ID NO: 8 is the amino acid sequence of the Musa acuminata PIN6 protein GSMUA_Achr8T08300.

SEQ ID NO: 9 is the amino acid sequence of the Vitis vinifera PIN6 protein GSVIVG01010025001.

SEQ ID NO: 10 is the amino acid sequence of the Solanum lycopersicum PIN6 protein Solyc06g059730.

SEQ ID NO: 11 is the amino acid sequence of the Populus trichocarpa PIN6 protein Potri.005G187500.1.

SEQ ID NO: 12 is the amino acid sequence of the Populus trichocarpa PIN6 protein Potri.002G072200.1.

SEQ ID NO: 13 is the amino acid sequence of the Oryza sativa PIN6 protein LOC_Os01g51780.1

SEQ ID NO: 14 is the amino acid sequence of the Zea mays PIN6 protein GRMZM5G839411.02.

SEQ ID NO: 15 is the amino acid sequence of the Arabidopsis thaliana PIN1 protein At1g73590.1.

SEQ ID NO: 16 is the amino acid sequence of the Arabidopsis thaliana PIN2 protein At5g57090.1.

SEQ ID NO: 17 is the amino acid sequence of the Arabidopsis thaliana PIN3 protein At1g70940.1.

SEQ ID NO: 18 is the amino acid sequence of the Arabidopsis thaliana PIN4 protein At2g01420.1.

SEQ ID NO: 19 is the amino acid sequence of the Arabidopsis thaliana PII5 protein At5g16530.1.

SEQ ID NO: 20 is the amino acid sequence of the Arabidopsis thaliana PINT protein At1g23080.1.

SEQ ID NO: 21 is the amino acid sequence of the Arabidopsis thaliana PIN8 protein At5g15100.1.

### DETAILED DESCRIPTION

### PIN6

Improving crop yield from agriculturally significant plants has become increasingly important. In addition to the growing need for agricultural products to feed, clothe and provide energy for a growing human population, climate-related effects and pressures are predicted to reduce the amount of arable land available for farming. These factors have led to grim forecasts of food security, particularly in the absence of major improvements in plant biotechnology and agronomic practices. In light of these pressures, environmentally sustainable improvements in technology, agricultural techniques, and pest management are vital tools to expand crop production on the limited amount of arable land available for farming.

Two major pressures that many of the world's farmers face today are nitrogen-deficient soils and drought, each of which can result in low yield or plant death. Symbiotic bacteria can improve plant biomass under low-nitrogen conditions. For example, legumes are known to form a symbiotic relationship with nitrogen-fixing rhizobia, generally referred to as root nodule symbiosis (RNS). The formation of root nodules is a hallmark of the symbiotic relationship between legumes and rhizobia.

Auxins are plant hormones that are thought to play a pivotal role in root nodule development. Auxins are synthesized in the apical tissues and demonstrate the ability to be accumulated in distant organs. Polar cell-to-cell auxin transport is mediated by carrier proteins of the AUX1 (Auxin resistant 1), LAX (like-AUX1), PGP (Phospho-glycoprotein) and PIN (PINFORMED) families. While AUX1, LAX and certain PGP-type proteins are involved in auxin import, PIN proteins mediate auxin export from the cell. That is, PIN-FORMED (PIN) efflux transporters are required for tissue-specific directional auxin transport and cellular auxin homeostasis.

The role of polar auxin transporters, PIN proteins, in root growth and lateral root development has been previously investigated. And more recent investigations have focused on the possible contributions of PIN proteins in the morphogenesis of the L. japonicus determinate root nodules, but these studies aimed to analyze expression levels and spatial expression patterns of PIN genes within their native legume (L. japonicus) root tissues. Moreover, such studies provide no suggestion that expression of PIN proteins in non-legume plants could be advantageous, let alone increase drought resistance or promote the formation of nodule-like structures in the presence of Rhizobia. Proteins involved in the formation of nodule-like structures in the presence of Rhizobia are therefore provided herein, which may be operably linked with regulatory elements for advantageous spatial and temporal expression of such proteins.

In Arabidopsis, the PIN protein family provides essential transport machinery to control auxin efflux and exhibit molecular divergence in their localization to either the plasma membrane (PM) (e.g., PIN1, 2, 3, 4, 7) or endoplasmic reticulum (ER) (e.g., PIN 5, 6, 8). All PIN proteins have a similar structure with amino- and carboxy-terminal hydrophobic, membrane-spanning domains separated by a central hydrophilic domain. The structure of the hydrophobic domains is well conserved. The hydrophilic domain is more divergent and it determines the eight groups within the protein family. The activity of PIN proteins is regulated at multiple levels, including transcription, protein stability, subcellular localization and transport activity. Different endogenous and environmental signals can modulate PIN activity and thus modulate auxin-distribution-dependent development.

In particular, the expression of PIN6 has been shown to be developmentally regulated exhibiting cell-type-, tissue- and organ-specific expression patterns and affecting auxin-dependent root growth and reproductive development. The subcellular localization of PIN6 may also be determined post-translationally by its phosphorylation status. Due to the many roles auxin plays in determining plant physiology, and the tightly controlled expression and regulation of auxin related transport proteins (e.g. PIN, AUX1, LAX and certain PGP-type proteins) it would not be expected that overexpression of a single auxin efflux transporter, PIN6, would yield the formation of nodule-like structures in non-legume plants in the presence of Rhizobia. Further surprising is the formation of nodule-like structures in non-host plants or plant parts, wherein said plants do not normally form a symbiotic relationship with nitrogen-fixing rhizobia. However, surprisingly, as demonstrated herein, PIN6 overexpression can induce nodule organogenesis by promoting the formation of nodule-like structures in the presence of Rhizobia. Similarly, it was also unexpectedly found that PIN6 overexpression in plants can yield increased drought resistance, as demonstrated herein. The instant disclosure therefore provides recombinant DNA molecules comprising PIN6, or other PIN6-related proteins, for example any of SEQ ID NOs: 3 and 5-14, operably linked to a heterologous promoter. Plants heterologously expressing or overexpressing PIN6, or other PIN6-related proteins, for example any of SEQ ID NOs: 3 and 5-14, which induce nodule organogenesis by promoting the formation of nodule-like structures in the presence of Rhizobia are further provided. Additionally, plants heterologously expressing or overexpressing PIN6, or other PIN6-related proteins, for example any of SEQ ID NOs: 3 and 5-14, which exhibit increased drought resistance are further provided.

The instant disclosure further provides recombinant DNA molecules as well as plants, plant cells, plant parts, or seeds comprising recombinant DNA molecules for expression of PIN6, or other PIN6-related proteins. In certain embodiments, recombinant DNA molecules for expression of any of SEQ ID NOs: 3 and 5-14, or variants or fragments thereof are provided. When expressed such sequences can promote the formation of nodule-like structures in the presence of rhizobia; and provide resistance to drought under extended water deprivation.

### Symbiotic Bacteria

The present disclosure provides DNA molecules encoding proteins that when expressed in a plant may promote the formation of nodule-like structures, a hallmark of root nodule symbiosis, in the presence of Rhizobia. Briefly, Rhizobia are bacteria found in soil that infect the roots of legumes to form root nodules which are involved in nitrogen utilization. As used herein, “rhizobia” refers to any bacteria that fix nitrogen after becoming established inside root nodules.

Symbiotic bacteria can be used with plants comprising the recombinant DNA molecules described herein to produce improved agronomic effects including improved plant growth or increased yield or biomass under reduced nitrogen conditions. Symbiotic bacteria useful with the disclosed plants include, but are not limited to Mesorhizobium loti, Sinorhizobium meliloti, Sinorhizobium fredii, Rhizobium sp. IRBG74 and NGR234, Bradyrhizobium sp., Azorhizobium caulinodans ORS571.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, a DNA molecule that comprises a synthetic DNA sequence or a DNA molecule that has been incorporated into a host cell's DNA by genetic transformation or gene editing.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

A polynucleotide or polypeptide provided herein may further include two or molecules which are heterologous with respect to one another. As used herein, the term “heterologous” refers to the combination of two or more polynucleotide molecules or two or more polypeptide molecules when such a combination is not normally found in nature. For example, the two molecules may be derived from different species and/or the two molecules may be derived from different genes, e.g. different genes from the same species or the same genes from different species. In some examples, a promoter is heterologous with respect to an operably linked transcribable polynucleotide molecule if such a combination is not normally found in nature, i.e. that transcribable polynucleotide molecule is not naturally occurring operably linked in combination with that promoter molecule.

Any number of methods well known to those skilled in the art can be used to isolate, manipulate, and detect a DNA molecule, or fragment thereof, disclosed herein. For example, PCR (polymerase chain reaction) technology can be used to amplify a particular starting DNA molecule and/or to produce variants of the original molecule. DNA molecules, or fragments thereof, can also be obtained by other techniques such as by directly synthesizing the fragment by chemical means, as is commonly practiced by using an automated oligonucleotide synthesizer.

Furthermore, DNA molecules of the present disclosure can be used in combination with methods known in the art to generate probes capable of detecting the presence of recombinant nucleic acid molecules of the present disclosure. As used herein, a “probe” is a nucleic acid sequence that is complementary to a strand of a target nucleic acid and useful in hybridization detection methods. Such sequences may hybridize specifically to a target DNA sequence under stringent hybridization conditions.

Stringent hybridization conditions are known in the art and described in, for example, MR Green and J Sambrook, Molecular cloning: a laboratory manual, 4th Edition, Cold Spring Harbor Laboratory Press, Cold Spring Harbor, N.Y. (2012). As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

As used herein, the term “percent sequence identity,” “percent identity,” or “% sequence identity” refers to the percentage of identical nucleotides or amino acids in a linear polynucleotide or polypeptide sequence of a reference (e.g. “query”) sequence (or its complementary strand) as compared to a test (e.g. “subject”) sequence (or its complementary strand) when the two sequences are optimally aligned. An optimal sequence alignment is created by manually aligning two sequences, e.g. a reference sequence and another sequence, to maximize the number of nucleotide matches in the sequence alignment with appropriate internal nucleotide insertions, deletions, or gaps. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar, Inc., 1228 S. Park St., Madison, Wis. 53715), and MUSCLE (version 3.6) (RC Edgar, Nucleic Acids Research (2004) 32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. As used herein, the term “sequence identity” refers to the extent to which two optimally aligned polynucleotide sequences or two optimally aligned polypeptide sequences are identical. As used herein, the term “reference sequence,” for example, may refer to a sequence provided as the polynucleotide sequences of any of SEQ ID NOs: 1, 2, and 4, or the polypeptide sequences of any of SEQ ID NOs: 3 and 5-14.

Thus, one embodiment of the disclosure is a recombinant DNA molecule comprising a sequence that when optimally aligned to a reference sequence, provided herein as the polynucleotide sequences of SEQ ID NOs: 1, 2, and 4, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example, the activity of any of SEQ ID NOs: 1, 2, and 4.

Similarly, another embodiment of the disclosure is a polypeptide molecule comprising a sequence that when optimally aligned to a reference sequence, provided herein as the polypeptide sequences of SEQ ID NOs: 3 and 5-14, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments, such sequences may be defined as having the activity of the reference sequence, for example, the activity of any of SEQ ID NOs: 3 and 5-14.

Also provided are fragments of polynucleotide sequences provided herein, for example fragments of a polynucleotide sequence selected from SEQ ID NOs: 1, 2, and 4. In specific embodiments, fragments of a polynucleotide sequences are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule of any of SEQ ID NOs: 1, 2, and 4. Methods for producing such fragments from a starting molecule are well known in the art. Fragments of a polynucleotide sequence provided herein may comprise the activity of the base sequence.

Recombinant polynucleotide sequences encoding fragments of polypeptide sequences provided herein are further envisioned, including polynucleotide sequences encoding fragments of a polypeptide sequence selected from SEQ ID NOs: 3 and 5-14. In specific embodiments, fragments of a polypeptide are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous amino acids, or longer, of a polypeptide molecule selected from SEQ ID NOs: 3 and 5-14. Methods for producing such fragments from a starting molecule are well known in the art. Fragments of a polynucleotide sequence provided herein may maintain the activity of the base sequence.

### Transcribable Polynucleotide Molecules

Recombinant DNA molecules provided herein include transcribable polynucleotide molecules or sequences encoding useful polypeptide sequences. In certain examples, transcribable polynucleotide molecules include sequences encoding PIN6, or PIN6-related polypeptides. Transcribable polynucleotides provided herein include SEQ ID NO: 1, 2, and 4, or polynucleotide sequences encoding any of SEQ ID NOs: 3 and 5-14, or fragments or variants thereof.

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

With respect to polypeptide sequences, the term “variant” as used herein refers to a second polypeptide sequence that is in composition similar, but not identical to, a first polypeptide sequence and yet the second polypeptide sequence still maintains the general functionality, i.e. same or similar activity, of the first polypeptide sequence. A variant may be a shorter or truncated version of the first polypeptide sequence and/or an altered version of the sequence of the first polypeptide sequence, such as one with different amino acid deletions, substitutions, and/or insertions. Variants having a percent identity to a sequence disclosed herein may have the same activity as the base sequence. For example, the transcribable polynucleotide molecule can encode a protein or variant of a protein or fragment of a protein that is functionally defined to maintain activity in transgenic host cells including plant cells, plant parts, explants and whole plants.

Similarly, with respect to polynucleotide sequences, the term “variant” as used herein refers to a second polynucleotide sequence that is in composition similar, but not identical to, a first polynucleotide sequence and yet the second polynucleotide sequence still maintains the general functionality, i.e. same or similar activity, of the first polynucleotide sequence. A variant may be a shorter or truncated version of the first polynucleotide sequence and/or an altered version of the sequence of the first polynucleotide sequence, such as one with different nucleotide deletions, substitutions, and/or insertions. Variants having a percent identity to a sequence disclosed herein may have the same activity as the base sequence. For example, variant polynucleotides may encode the same or a similar protein sequence or have the same or similar gene regulatory activity as the base sequence.

As used herein, “modulation” of expression refers to the process of effecting either overexpression or suppression of a polynucleotide or a protein.

As used here, the term “overexpression” as used herein refers to an increased expression level of a polynucleotide or a protein in a plant, plant cell, or plant tissue, compared to expression in a wild-type plant, cell, or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell, or tissue.

Overexpression can be achieved using numerous approaches. In one embodiment, overexpression can be achieved by placing the DNA sequence encoding one or more polynucleotides or polypeptides under the control of a promoter, examples of which include but are not limited to endogenous promoters, homologous promoters, heterologous promoters, inducible promoters, development specific promoters, and tissue specific promoters. In one exemplary embodiment, the promoter is a constitutive promoter, for example, the cauliflower mosaic virus 35S promoter and other constitutive promoters known in the art. In another embodiment, the promoter is an auxin-inducible promoter, a root-specific promoter, a drought inducible promoter, or fragments or variants thereof. Thus, depending on the promoter used, overexpression can occur throughout a plant, in specific tissues of the plant, in specific stages of development of the plant, or in the presence or absence of different inducing or inducible agents, such as hormones or environmental signals.

In certain embodiments, the expression or overexpression of a transcribable polynucleotide molecule encoding a protein as disclosed herein can effect an enhanced trait or altered phenotype directly or indirectly. In some cases, it may do so, for example, by inducing nodule organogenesis. In an exemplary embodiment, the protein produced from the transcribable polynucleotide molecule can promote the formation of nodule-like structures. In certain exemplary embodiments, the protein produced from the transcribable polynucleotide molecule can increase drought tolerance.

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, product, nutritional profile, disease, or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism.

In one embodiment of the disclosure, a promoter is incorporated into a construct such that the promoter is operably linked to a transcribable polynucleotide molecule that encodes a PIN6 or a PIN6-related protein, including any of SEQ ID NOs: 3 and 5-14 or fragments or variants thereof. The expression of the transcribable polynucleotide molecule is desirable in order to confer an agronomically beneficial trait, including but not limited to increased drought resistance. An agronomically beneficial trait may also be, for example, modified yield, improved plant growth and development, improved biomass, increased resistance to environmental stress (e.g. nitrogen limited conditions), improved nitrogen fixation, improved fungal disease resistance, improved virus resistance, improved nematode resistance, improved bacterial disease resistance, improved starch production, modified oil production, modified fatty acid content, improved protein production, improved fruit ripening, enhanced animal and human nutrition, improved seed production, improved fiber production, and improved biofuel production. In certain embodiments, the increased resistance to environmental stress (e.g. nitrogen limited conditions) or improved nitrogen fixation is a product of formation of nodule-like structures induced by the expression of the transcribable polynucleotide molecule.

Transcribable polynucleotide molecules may also be markers useful in detecting transformed plant cells, plant tissue, plant parts, or plants described herein. As used herein the term “marker” refers to any transcribable polynucleotide molecule whose expression, or lack thereof, can be screened for or scored in some way. Marker genes for use in the practice of the present disclosure include, but are not limited to transcribable polynucleotide molecules encoding β-glucuronidase (GUS described in U.S. Pat. No. 5,599,670), green fluorescent protein and variants thereof (GFP described in U.S. Pat. Nos. 5,491,084 and 6,146,826), proteins that confer antibiotic resistance, or proteins that confer herbicide tolerance. Useful antibiotic resistance markers, including those encoding proteins conferring resistance to kanamycin (nptII), hygromycin B (aph IV), streptomycin or spectinomycin (aad, spec/strep) and gentamycin (aac3 and aacC4) are known in the art. Herbicides for which transgenic plant tolerance has been demonstrated and the method of the present disclosure can be applied, include, but are not limited to: amino-methyl-phosphonic acid, glyphosate, glufosinate, sulfonylureas, imidazolinones, bromoxynil, dalapon, dicamba, cyclohexanedione, protoporphyrinogen oxidase inhibitors, and isoxasflutole herbicides.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g, alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S). Other possible selectable marker genes will be apparent to those of skill in the art and are encompassed by the present disclosure.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell.

The constructs of the present disclosure may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present disclosure.

Methods are known in the art for assembling and introducing constructs into a cell in such a manner that the transcribable polynucleotide molecule is transcribed into a functional mRNA molecule that is translated and expressed as a protein product. For the practice of the present disclosure, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, Fla. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those disclosed herein such or known in the art, or variants or fragments thereof. Any such regulatory elements may be provided in combination with other regulatory elements. Such combinations can be designed or modified to produce desirable regulatory features. In one embodiment, constructs of the present disclosure comprise at least one regulatory element operably linked to a transcribable polynucleotide molecule operably linked to a 3′ UTR.

Constructs of the present disclosure may include any promoter or fragment or variant thereof provided herein, or known in the art. For example, a transcribable polynucleotide sequence provided herein, such as sequences encoding one or more polypeptides selected from SEQ ID NOs: 3 and 5-14, or variants or fragments thereof, may be operably linked to, a heterologous promoter such as the Cauliflower Mosaic Virus 35S transcript promoter (see, U.S. Pat. No. 5,352,605), an auxin-inducible promoter (e.g. GH3, SAUR, Nt103-1, ARFs, or AUX/IAAs), a root-specific promoter (e.g. Os03g01700, Os02g37190, SRSP, IDE1, IDE2, IDS2, Sad1, or Sad2) or a drought inducible promoter (e.g. Rab21, Wsi18, Lea3, Uge1, Dip1, R1G1B, ERD1, RGLG1, RGLG2, DREB2A, DRIPs, MYC2).

A construct provided herein may further comprise additional elements useful in regulating or modulating expression of a transcribable polynucleotide, including leader, enhancer, intron, and 3′ UTR sequences. A construct provided herein may further comprise one or more marker sequences for identification of the construct in plant cells, plant tissue, or plants.

### Regulatory Elements

A regulatory element is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a regulatory element may be comprised of expression elements, such as enhancers, promoters, and introns, operably linked. A regulatory element may also be comprised of leaders and 3′ untranslated regions (3′ UTRs). Regulatory elements, capable of providing a unique spatial and temporal expression profile to an operably linked heterologous transcribable polynucleotide molecule are therefore useful for modifying plant phenotypes through the methods of genetic engineering.

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, the term “expression pattern” or “expression profile” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules.

In specific embodiments of the disclosure, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription. In accordance with the disclosure a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants of the promoter having a similar expression pattern to the original promoter.

As used herein, the term “chimeric” refers to a single DNA molecule produced by fusing a first DNA molecule to a second DNA molecule, where neither the first nor second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present disclosure.

### Plants Comprising DNA Molecules

Constructs, expression cassettes, and vectors comprising DNA molecules as disclosed herein can be constructed and introduced into a plant cell in accordance with transformation methods and techniques known in the art. For example, Agrobacterium-mediated transformation is described in U.S. Patent Application Publications 2009/0138985A1 (soybean), 2008/0280361A1 (soybean), 2009/0142837A1 (corn), 2008/0282432 (cotton), 2008/0256667 (cotton), 2003/0110531 (wheat), 2001/0042257 A1 (sugar beet), U.S. Pat. No. 5,750,871 (canola), U.S. Pat. No. 7,026,528 (wheat), and U.S. Pat. No. 6,365,807 (rice), and in Arencibia et al. (1998) Transgenic Res. 7:213-222 (sugarcane) all of which are incorporated herein by reference in their entirety. Transformed cells can be regenerated into transformed plants that express the polypeptides disclosed herein and demonstrate activity through bioassays as described herein as well as those known in the art. Plants can be derived from the plant cells by regeneration, seed, pollen, or meristem transformation techniques. Methods for transforming plants are known in the art.

The term “plant cell” or “plant” can include but is not limited to a dicotyledonous or monocotyledonous plant. In certain embodiments, plants provided herein are legumes, including, but not limited to, beans, soybeans, peas, chickpeas, peanuts, lentils, lupins, mesquite, carob, tamarind, alfalfa, and clover. Plants provided herein may also be non-legume plants.

The term “plant cell” or “plant” can also include but is not limited to an alfalfa, banana, barley, bean, broccoli, cabbage, brassica (e.g canola), carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, legumes, non-legumes, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, root stocks, rye, safflower, shrub, sorghum, Southern pine, soybean, spinach, squash, strawberry, sugar beet, sugarcane, sunflower, corn (i.e. maize, such as sweet corn or field corn, sweet gum, sweet potato, switchgrass, tea, tobacco, tomato, triticale, turf grass, watermelon, and wheat plant cell or plant.

In certain embodiments, transgenic plants and transgenic plant parts regenerated from a transgenic plant cell are provided. In certain embodiments, the transgenic plants can be obtained from a transgenic seed, by cutting, snapping, grinding or otherwise disassociating the part from the plant. In certain embodiments, the plant part can be a seed, a boll, a leaf, a flower, a stem, a root, or any portion thereof, or a non-regenerable portion of a transgenic plant part. As used in this context, a “non-regenerable” portion of a transgenic plant part is a portion that cannot be induced to form a whole plant or that cannot be induced to form a whole plant that is capable of sexual and/or asexual reproduction. In certain embodiments, a non-regenerable portion of a plant part is a portion of a transgenic seed, boll, leaf, flower, stem, or root.

The term “transformation” refers to the introduction of a DNA molecule into a recipient host. As used herein, the term “host” refers to bacteria, fungi, or plants, including any cells, tissues, organs, or progeny of the bacteria, fungi, or plants. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA molecule. The introduced DNA molecule may also be transiently introduced into the recipient cell such that the introduced DNA molecule is not inherited by subsequent progeny. The term “transgenic” refers to a bacterium, fungus, or plant containing one or more heterologous DNA molecules.

There are many methods well known to those of skill in the art for introducing DNA molecules into plant cells. The process generally comprises the steps of selecting a suitable host cell, transforming the host cell with a vector, and obtaining the transformed host cell. Methods and materials for transforming plant cells by introducing a plant construct into a plant genome in the practice of this technology can include any of the well-known and demonstrated methods. Suitable methods include, but are not limited to, bacterial infection (e.g., Agrobacterium), binary BAC vectors, direct delivery of DNA (e.g., by PEG-mediated transformation, desiccation/inhibition-mediated DNA uptake, electroporation, agitation with silicon carbide fibers, and acceleration of DNA coated particles), gene editing (e.g., CRISPR-Cas systems), among others.

Host cells may be any cell or organism, such as a plant cell, algal cell, algae, fungal cell, fungi, bacterial cell, or insect cell. In specific embodiments, the host cells and transformed cells may include cells from crop plants.

A transgenic plant subsequently may be regenerated from a transgenic plant cell described herein. Using conventional breeding techniques or self-pollination, seed may be produced from this transgenic plant. Such seed, and the resulting progeny plant grown from such seed, will contain the recombinant DNA molecule described herein, and therefore will be transgenic.

Transgenic plants can be self-pollinated to provide seed for homozygous transgenic plants (homozygous for the recombinant DNA molecule) or crossed with non-transgenic plants or different transgenic plants to provide seed for heterozygous transgenic plants described herein (heterozygous for the recombinant DNA molecule). Both such homozygous and heterozygous transgenic plants are referred to herein as “progeny plants.” Progeny plants are transgenic plants descended from the original transgenic plant and containing the recombinant DNA molecule described herein. Seeds produced using a transgenic plant described herein can be harvested and used to grow generations of transgenic plants, i.e., progeny plants, comprising the construct described herein and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory elements disclosed herein. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA molecule can be measured using TaqMan® (Applied Biosystems, Foster City, Calif.) reagents and methods as described by the manufacturer and PCR cycle times determined using the TaqMan® Testing Matrix. Alternatively, the Invader® (Third Wave Technologies, Madison, Wis.) or SYBR Green (Thermo Fisher, A46012) reagents and methods as described by the manufacturer can be used to evaluate transgene expression.

The seeds of the plants of disclosed herein can be harvested from fertile transgenic plants and be used to grow progeny generations of transformed plants of this disclosure including hybrid plant lines comprising the constructs described herein and expressing a gene of agronomic interest.

The present disclosure also provides for parts of the plants of the present disclosure. Plant parts, without limitation, include leaves, stems, roots, tubers, seeds, endosperm, ovule, and pollen. The present disclosure also includes and provides transformed plant cells which comprise a nucleic acid molecule.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immuno-precipitation, and ELISA.

As an alternative to traditional transformation methods, a DNA molecule, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

### Commodity Products

The present disclosure provides a commodity product comprising DNA molecules. As used herein, a “commodity product” refers to any composition or product which is comprised of material derived from a plant, seed, plant cell, or plant part comprising a DNA molecule. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animals consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the present disclosure can thus be used to manufacture any commodity product typically acquired from plants or parts thereof.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: Root Organogenesis Responses to Transient Auxin and Cytokinin Treatments

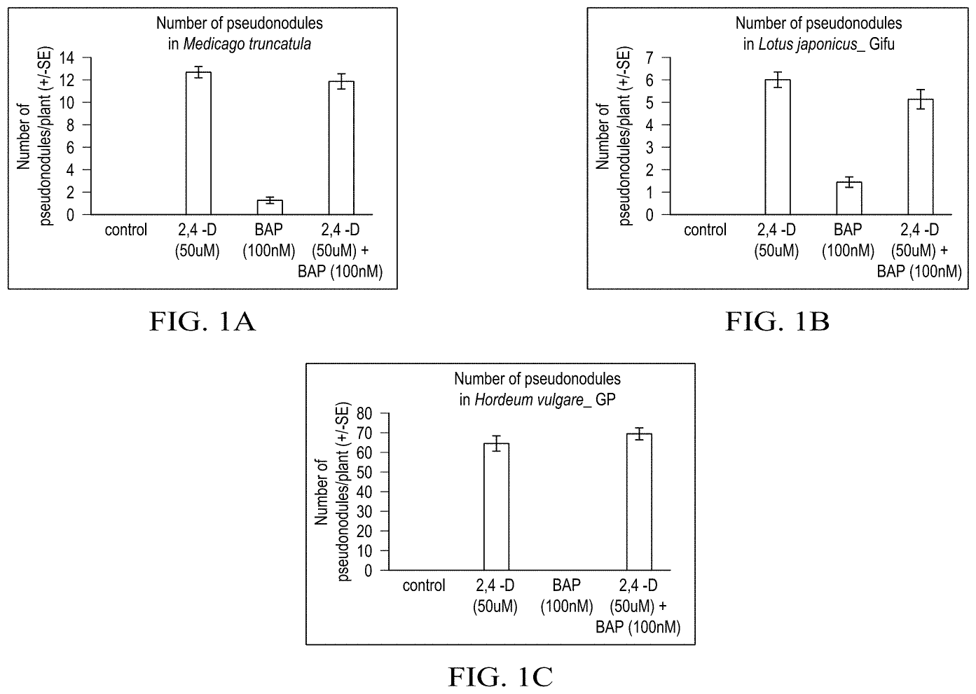

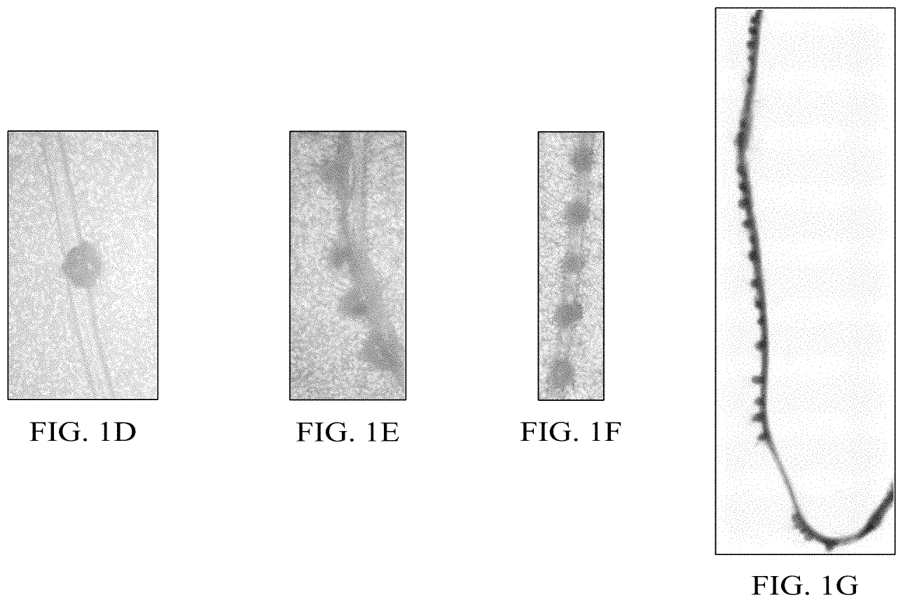

In order to investigate the potential impact of auxin and cytokinin treatment on root organogenesis, seeds of the Medicago truncatula cultivar Jester, the Lotus japonicus ecotype Gifu, and the Hordeum vulgare cultivar Golden Promise were sterilized, and then seedlings were grown on FP media (without Nitrogen and Phosphorus) for 3, 7, and 2 days, respectively. Grown plants were transformed to FP media with either control treatment, 50 μM of 2,4-Dichlorophenoxyacetic acid (synthetic auxin), 100 nM of 6-Benzylaminopurine (synthetic cytokinin) or both of the hormones for 1 hour before moving to FP media. FIG. 1A-1C show the number of pseudonodules/Arrested Lateral roots (ALR) counted 7 days after control treatment, treatment with 2,4-Dichlorophenoxyacetic acid (2,4-D), 6-Benzylaminopurine (BAP), or both. This demonstrates that synthetic auxin treatment leads to increased pseudonodule formation in Medicago truncatula, Lotus japonicus, and the Hordeum vulgare, compared with control plants.

As shown in FIG. 1D-G, auxin treatment leads to the formation of pseudonodules on barley (Hordeum vulgare) roots which appear independently and in clusters.

### Example 2: PIN6 Overexpression Impairs Lateral Root Growth

In order to investigate lateral root (LR) growth in plants over-expressing PIN6, Arabidopsis thaliana plant lines stably transformed with a construct comprising PIN6 and GFP under the control of the cauliflower mosaic virus 35S promoter were generated. Briefly, to generate Arabidopsis plants overexpressing PIN6, wild type Col-0 plants were transformed with a vector pMDC32 comprising the Arabidopsis PIN6 genomic sequence, fused to GFP or not fused to GFP, and under the control of the strong CaMV 35S promoter. In addition, another PIN6 over-expressor line was generated in which genomic PIN6 fused to GFP was driven by the Arabidopsis ubiquitin promoter. Additionally, to obtain barley plants conditionally overexpressing PIN6, we transformed WT (Golden promise) barley plants with a construct consisting of the Arabidopsis PIN6 genomic sequence either fused to GFP (ProZmOptXVE::PIN6-GFP) or not fused to GFP (ProZmOptXVE::PIN6-CDS), and driven by an optimized XVE (ß-estradiol inducible) promoter.

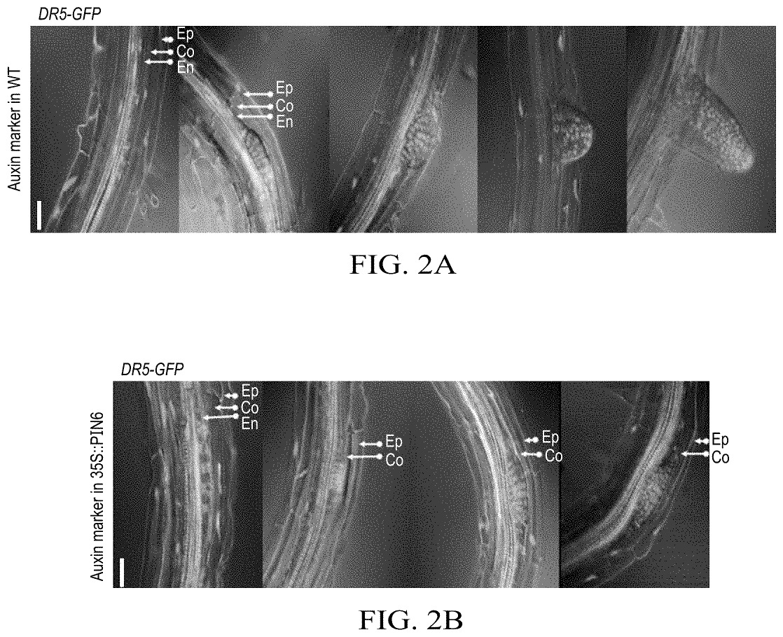

As shown in FIG. 2, lateral root growth was observed in WT plants (FIG. 2A). However, over-expression of the auxin efflux carrier PIN6 in Arabidopsis impaired lateral root growth (FIG. 2B).

### Example 3: Formation of Nodule-Like Structures in the Presence of Rhizobia

As described in Example 2, over-expression of the auxin efflux carrier PIN6 in Arabidopsis impairs lateral root growth. Interestingly, it has been shown that the beneficial bacteria Rhizobium sp. IRBG74 can rapidly (after 7 days) promote lateral root development in Arabidopsis, presumably via the auxin released by bacteria (Zhao et al. 2017). Therefore, the ability of IRBG74 to re-activate the development of Pro35S::PIN6 arrested lateral roots, as described in Example 2, was tested. Briefly, Arabidopsis seeds (Wild type Colombia-0 (WT-Col 0) and Pro35S::PIN6-GFP were surface sterilized by incubating the seeds in 2 ml 80% Ethanol for 2 min, followed by a quick wash in 100% Ethanol, and then dried on filter paper. Seeds were sown on solid Arabidopsis medium [2.3 g 1-1 Murashige and Skoog (MS) salts, 1% sucrose, 1.6% agar-agar (pH 6.0) adjusted with KOH]. After vernalization for 2 d at 4° C., germination took place under a long-day period with 16 h, 35 μmol/m2/s light and 8 h darkness, at 21° C. The Rhizobia inoculum was prepared using tetracycline resistant Rhizobium sp. (IRBG74), cultured in TY-medium (20 μg/ml) for 72 h at 28° C. were sub-cultured in TY-liquid medium for 24 h. Bacterial liquid culture was centrifuged at 4000 rpm for 10 min. Bacteria were re-suspended in 1 ml FP medium, 1 μCaCl2, 1 μM aminoethoxyvinylglycine (AVG, an ethylene biosynthesis inhibitor) and completed to 1 ml with SFP. Bacterial inoculum OD was adjusted to 0.01.

Seven (7) day old Arabidopsis seedlings were inoculated by soaking the root with liquid IRBG74 DsRed (OD 0.01) for 7 min. After removal of inoculum excess, plants were left to grow in conditions identical to those applied for seed germination (see directly above). Seedlings were inspected using a Zeiss stereomicroscope equipped with a fluorescence lamp. High magnification views were obtained using laser scanning microscopes (Zeiss LSM880 or Leica SP8). After one week of incubation, these bacteria restored LR development.

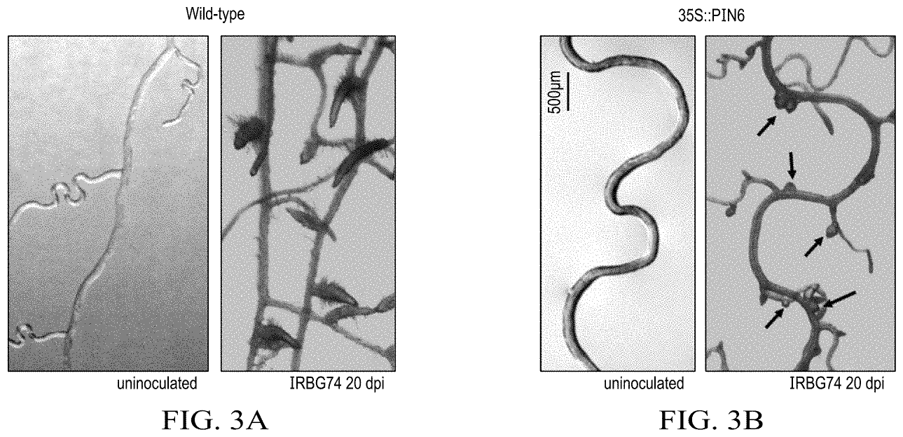

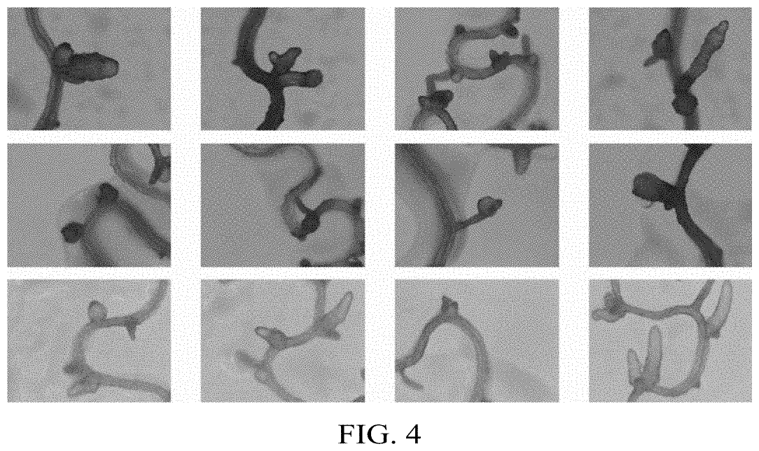

However, when the roots were inspected two additional weeks later, it was unexpectedly found that plants overexpressing PIN6 had developed round-shaped nodule-structures which were visually distinct from LRs observed in wild type plants in the presence of IRGB74 (FIGS. 3 and 4). This surprising result was particularly intriguing since IRBG74 is a true nitrogen fixing symbiont that nodulates several plant species including Sesbania rostrata and Lotus japonicus. These results indicate that PIN6 over-expression in the presence of Rhizobia can yield the formation of nodule-like structures in non-legume plants. As can be seen in FIG. 3, Pro35S::PIN6-mediated nodule-like structures (NDS) are round and bold (without root hairs) (FIG. 3B), whereas the majority of structures developed by wild type (Col-0) plants (FIG. 3A), after initially acquiring a round-shape, fail to maintain the nodule-like aspect and distally elongated an LR. These surprising results indicate a method to induce nodule organogenesis on non-host plants that comprises inoculating plants overexpressing PIN6 with a symbiotic bacteria, e.g. a Rhizobium.

### Example 4: PIN6/Rhizobia-Dependent Nodule-Like Structures Derive from Pericycle Cell Divisions

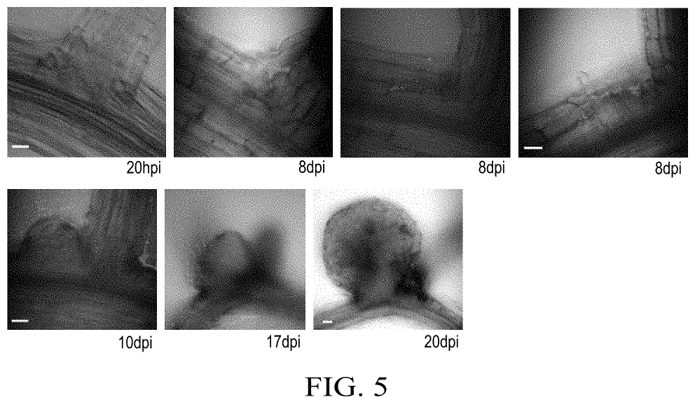

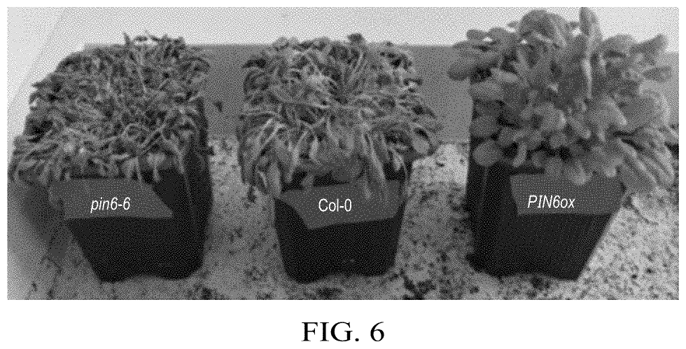

To further investigate the development of the nodule-like structures formed by PIN6 over-expressing plants in the presence of Rhizobia, IRBG74-DsRed localization was visualized spatially and temporally. These experiments demonstrated that IRB G74 takes advantage of the root breaches occurring during lateral root emergence to enter the root. The tissue-specific accumulation of bacteria coincides with the local activation of cell proliferation leading to the development of root-nodule like structures. As shown in FIG. 5, PIN6/IRBG74-dependent nodule-like structures derive from pericycle cell divisions.

### Example 5: Drought Resistance of PIN6 Overexpressing Plant Lines

Modulation of PIN6 expression in plants has been shown to regulate plant growth and development. Specifically, PIN6 was previously reported as a negative regulator of plant growth and development (Cazzonelli et al. 2013; Bender et al. 2013). Additionally, knock-out pin6 mutants grow relatively fast compared to WT plants (Ditengou et al. 2018); and Arabidopsis plants overexpressing PIN6 are dwarf and develop slowly (Cazzonelli et al. 2013; Simon et al. 2016; Ditengou et al. 2018).

However, during experiments aiming to characterize the role of PIN6 over-expression on plant development, it was unexpectedly found that plants overexpressing PIN6 are very resistant to drought when experiencing extended water deprivation (15 d). To investigate this phenotype, Arabidopsis seeds, WT-Col0, pin6 knock-out (pin6-6), and Pro35S::PIN6-GFP (PIN6OX) were surface sterilized by incubating the seeds in 2 ml 80% Ethanol for 2 min, followed by a quick wash in 100% Ethanol, and then dried on filter paper. Seeds were sown on solid Arabidopsis medium [2.3 g 1-1 Murashige and Skoog (MS) salts, 1% sucrose, 1.6% agar-agar (pH 6.0) adjusted with KOH]. After vernalization for 2 d at 4° C., germination took place under a long-day period with 16 h, 35 μmol/m²/s light and 8 h darkness, at 21° C. Forty-eight (48) hours vernalized Arabidopsis seeds (WT-Col0, pin6 knock-out and Pro35S::PIN6-GFP) were sown directly in soil and grown for 2 weeks. Then plants were exposed to drought conditions by non-watering for 2 additional weeks.

These experiments demonstrate a role for PIN6 as a component of drought tolerance regulation. Specifically, the results shown in FIG. 6 show that pin6 knock-out mutants (pin6-6) displayed more sensitivity to drought than WT control plants, while PIN6 over-expression can yield improved drought tolerance.

### Example 6: Growth and Root Phenotypic Analysis of PIN6 Expression in Barley

To test the impact of PIN6 expression in plants lacking an endogenous PIN6 gene, such as Poaceae, barley plants expressing the Arabidopsis PIN6 (and PIN6-GFP) driven by the ß-estradiol inducible ProZmOptXVE promoter were generated as described in Example 2 above. To transformants were first grown on soil under a long day period with 16 h (150 μmol/m²/s light), 8 h darkness at 21° C., in order to produce new plantlets. The latter were split into two sets, one being repotted and supplemented with fertilizers for seed production, whereas the second set was transferred to new pots containing sand+vermiculite (1/1), and inoculated with IRBG74 DsRed (OD=0.001) in 10 ml FP medium supplemented with 1 μM CaCl2 and 1 μM AVG. These plants were watered every 2 days with 50 ml sterile water with or without 1 μM ß-estradiol to induce PIN6 expression. Visual inspections and quantification of growth parameters were performed after 40 days. Root system architecture was imaged using a Zeiss stereomicroscope, whereas closer views of lateral roots and nodule-like structures were captured using a Zeiss microscope equipped with the Apotome module.

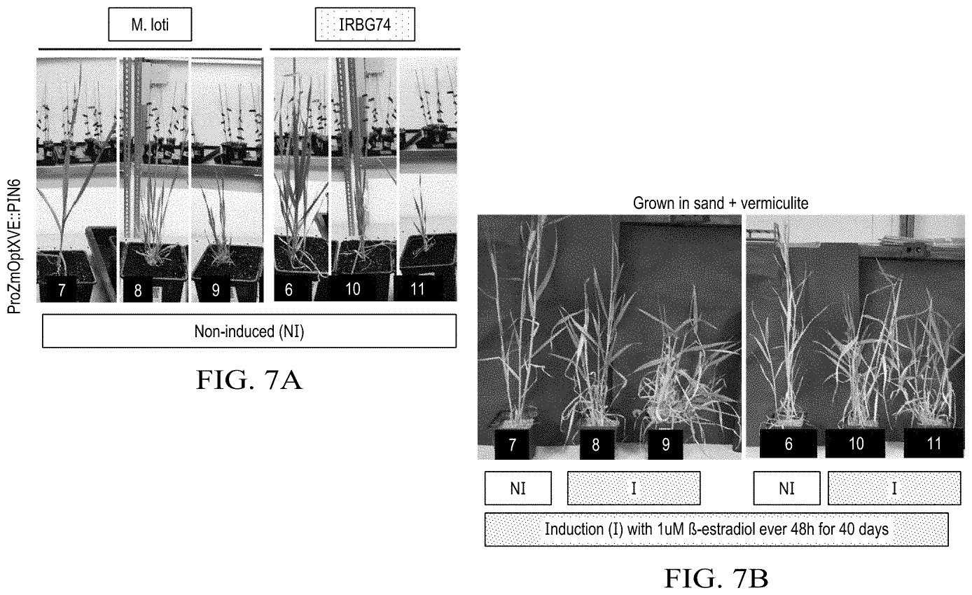

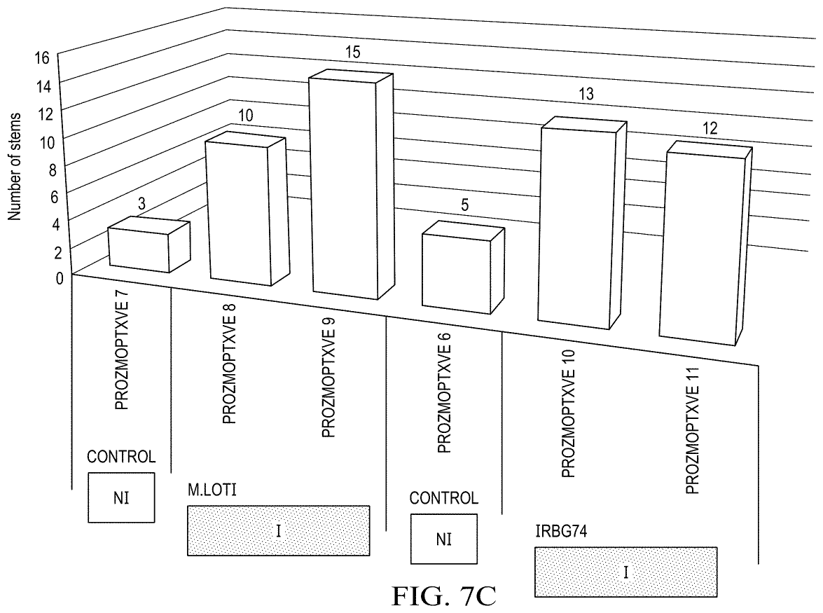

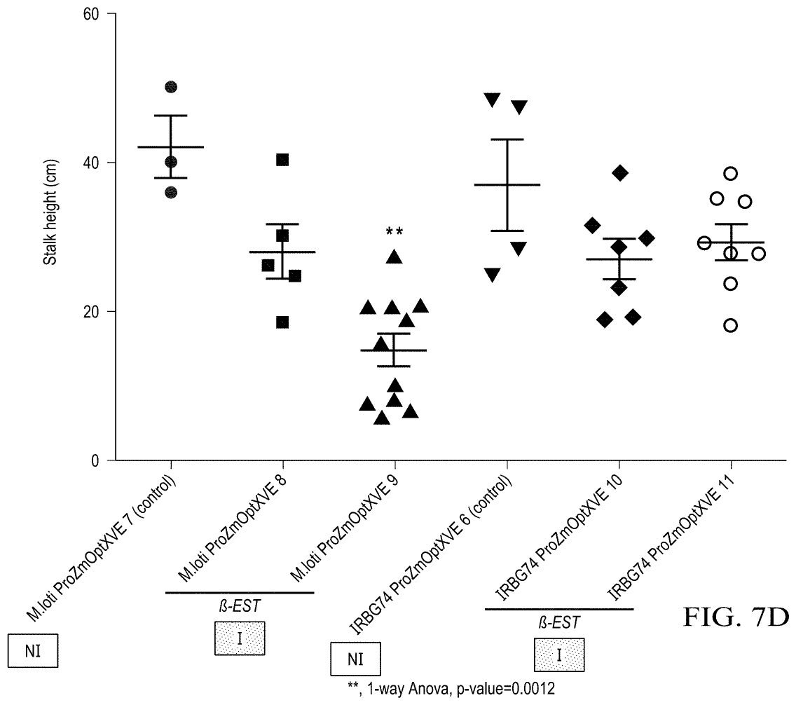

FIG. 7 shows plants expressing PIN6 under the control of a ß-estradiol-inducible promoter in the presence of Rhizobia (IRBG74), induced by addition of 1 μM ß-estradiol every 48 h for 40 days. When compared to non-induced control plants, watering with ß-estradiol strongly affected plant development. Induced plants developed significantly more stems; however, the stems were shorter than that of the non-induced plants.

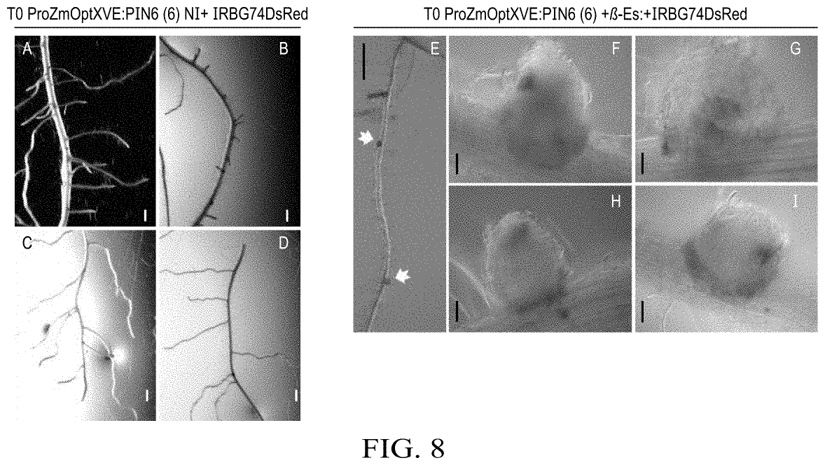

As shown in FIG. 8, further inspection of root systems of these barley plants showed the formation of nodule-like structures consistent with structures observed in Arabidopsis plants. Thus, these results indicate that PIN6 over-expression in the presence of Rhizobia in monocot plants also has the ability to yield nodule-like structures.

### Example 7: Drought Resistance of PIN6 Overexpressing Barley Lines

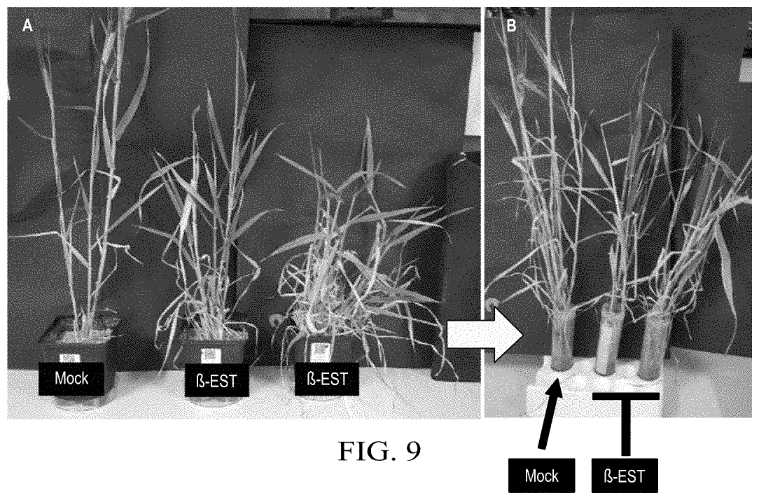

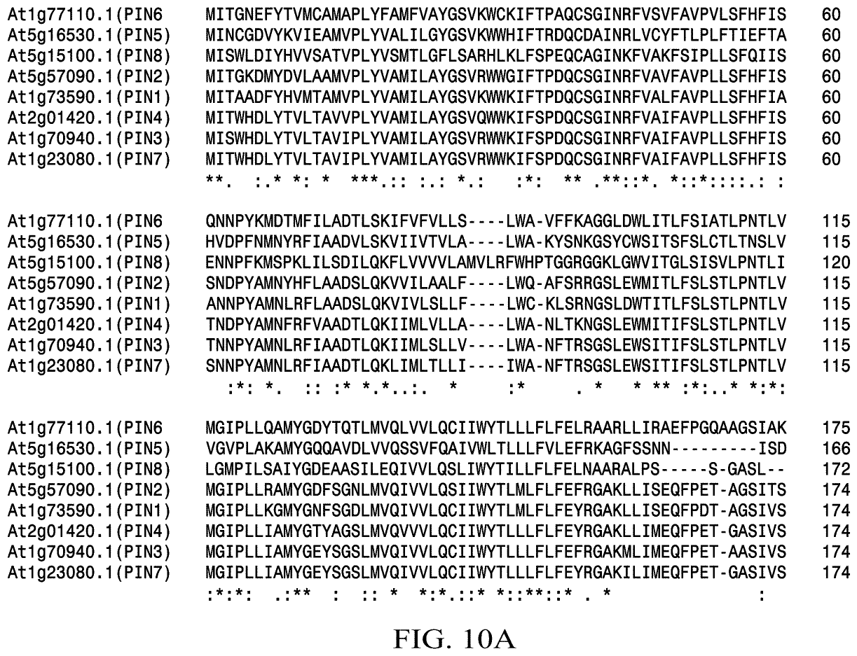

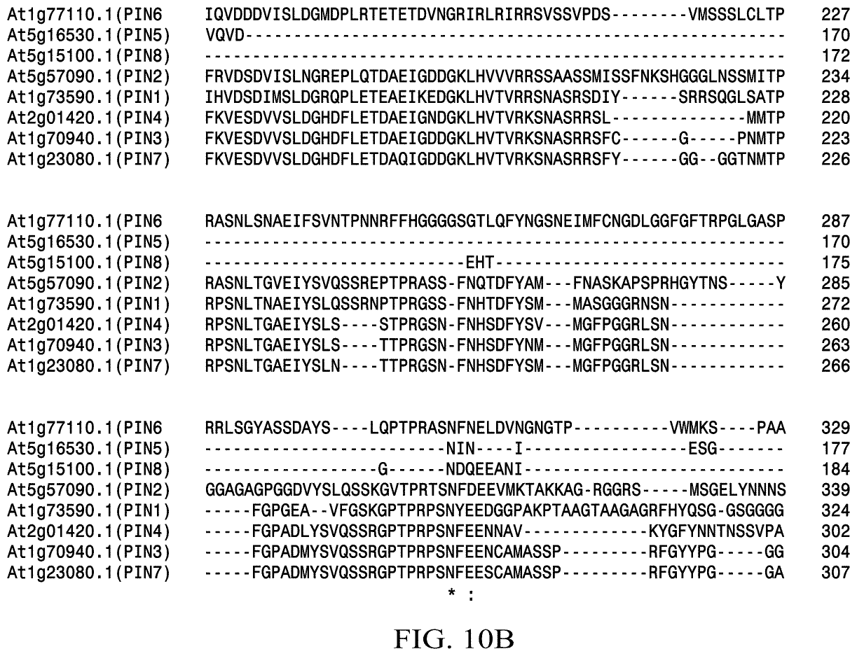

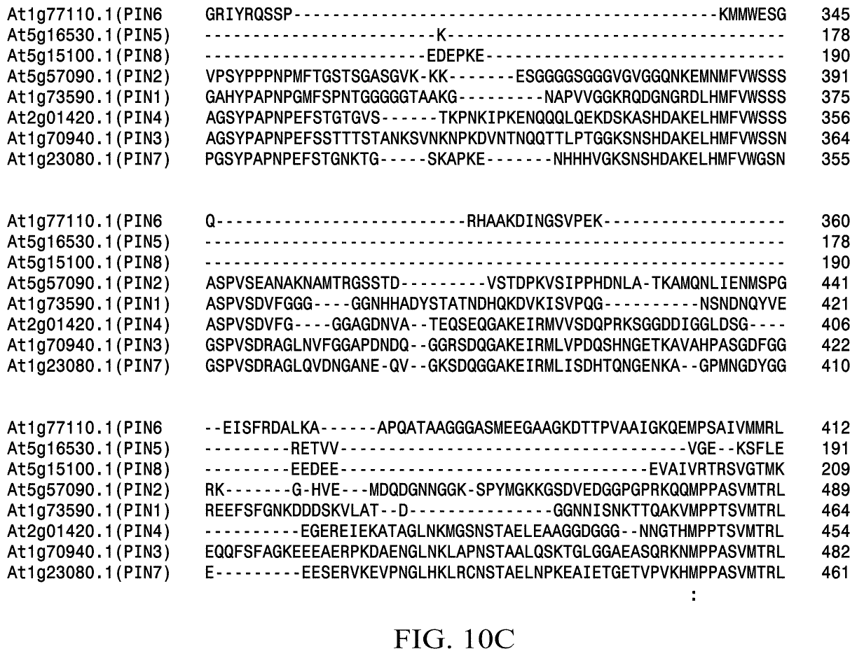

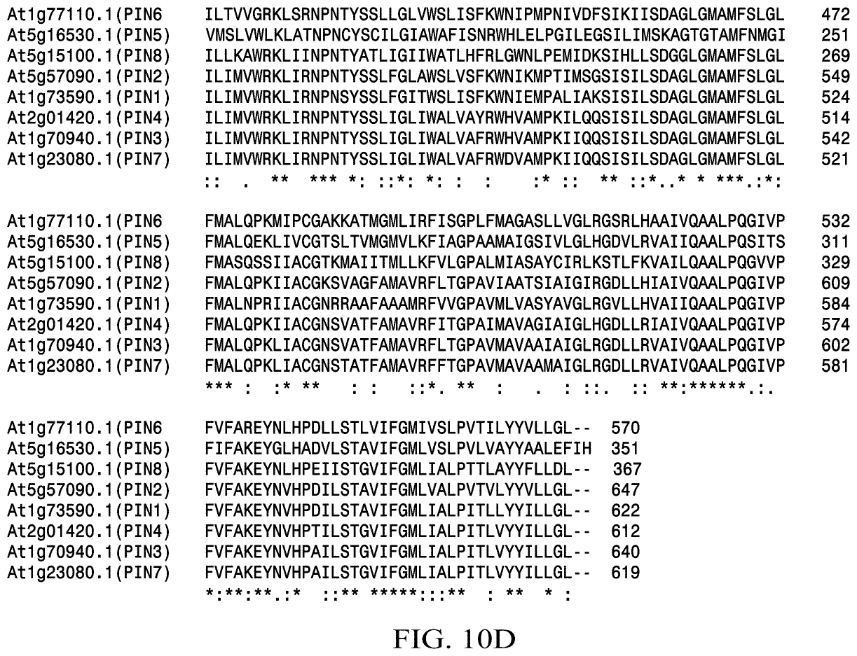

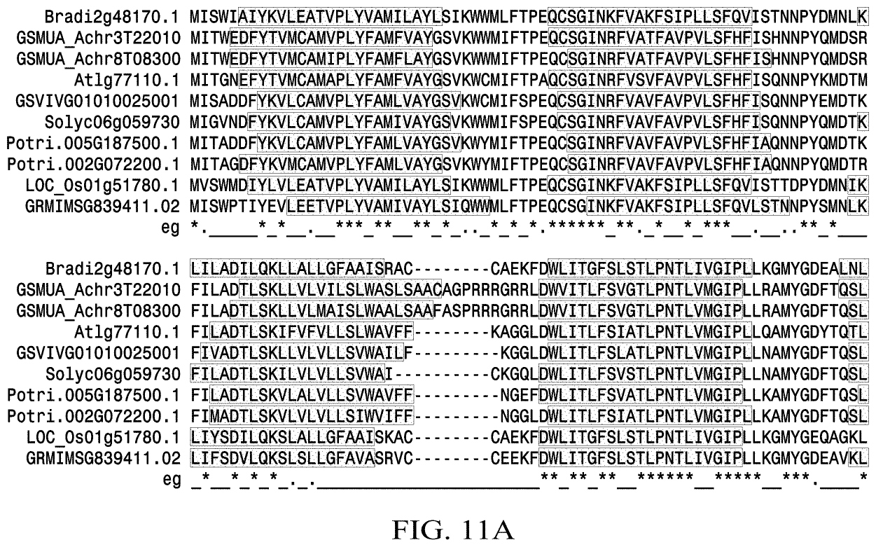

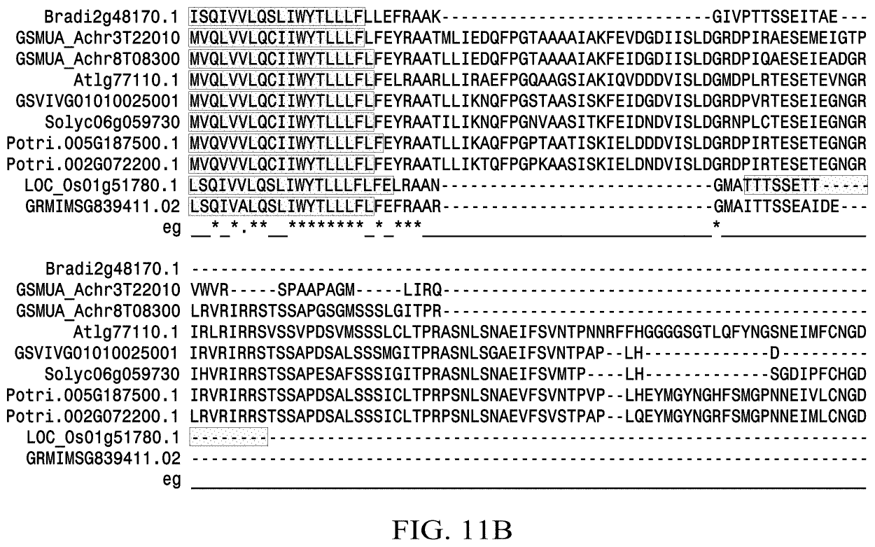

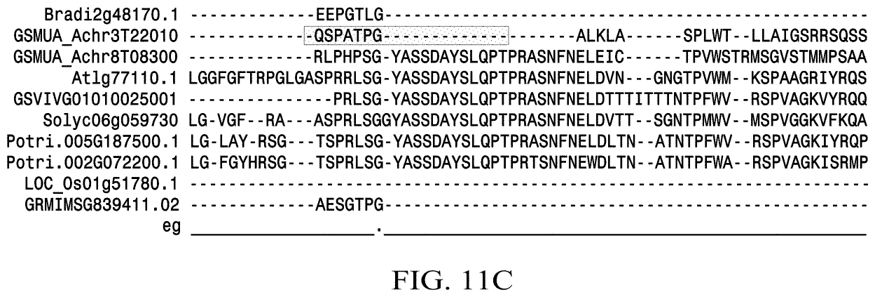

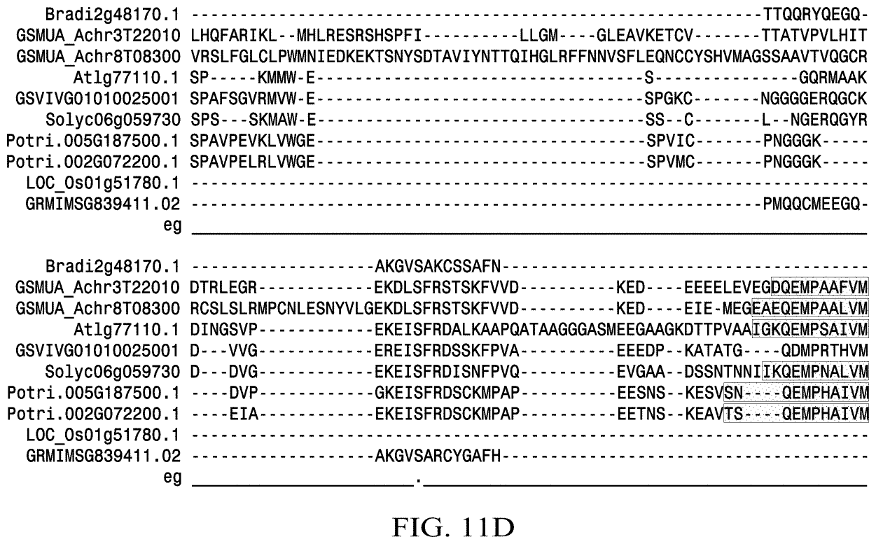

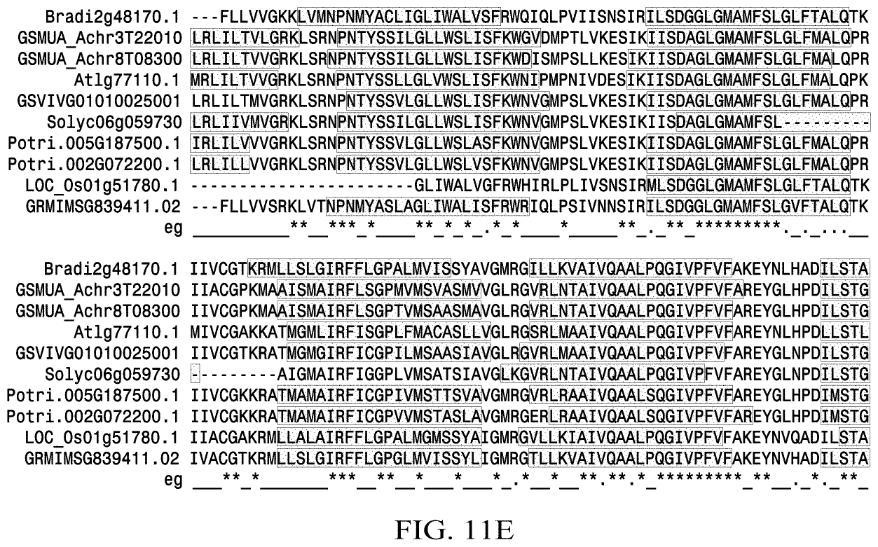

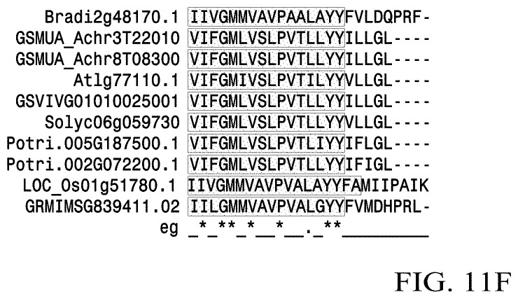

To test the impact of PIN6 overexpression on drought resistance in monocots, control, and ß-estradiol-induced barley plants as described in Example 6 were transferred to 50 ml water-filled Falcon tubes and grown for 2 weeks. As shown in FIG. 9, control plants consumed all water and were withered whereas ß-estradiol-induced plants were still green. This result suggests that overexpressing PIN6, in both dicots and monocots, increases drought tolerance.

### Example 8: Additional Phenotypic Analysis of PIN6 Expression in Barley

To further investigate the impact of PIN6 expression in monocots, barley plants expressing Arabidopsis or Lotus PIN6 (AtPIN6-GFP or LjPIN6-GFP) under the control of the ß-estradiol inducible ProZmOptXVE promoter or under the control of the Ubiquitin promoter are generated as described in Example 2 above.

Briefly, seeds of WT and the progeny of homozygous T₀ transformants are washed with 70% ethanol for 2 min, soaked in 6% sodium hypochlorite with 0:01% and Tween 80 for 2 h, and rinsed three times with sterile water. After manual removal of seed coat, seeds are vernalized on 1% agar at 4° C. in the dark for 48 h. To accelerate seed germination, seeds are exposed to light for 1 h and then incubated for 24 h in dark at 21° C. Thereafter they are exposed to light (under a long-day period with 16 h, 35 μmol/m²/s light and 8 h darkness) and allowed to develop for 48 h. PIN6 expression is quantified using both qRT-PCR and Western blot using a leaf fragment. Plants showing high PIN6 expression levels are transferred to pots containing sand+vermiculite and inoculated with IRBG74. Quantification of growth parameters, the presence/absence of nodule like structures, as well as PIN6 subcellular localization in roots is assessed after 40 days to assess plants for increased drought tolerance. Plants' ability to cope with water scarcity is tested as previously described herein. Briefly, growth parameters (root and shoot weight) of plants grown for 2 weeks in Falcon™ tubes (in water or water supplemented with ß-estradiol) will be quantified. This system allows plants to be quickly confronted to desiccation, as the liquid evaporates.

### Example 9: Phenotypic Analysis of PIN6 Expression in Potato

To test the impact of PIN6 overexpression in potato, potato roots are streaked into LB Agar plates containing Agrobacterium rhizogenes strain Arqua1 harboring a ProAtUb10::AtPIN6 construct. The inoculated potato seedlings are placed on ½ MS medium plates including vitamins plus 3% sucrose, and the hypocotyl is covered with wet filter paper for one week. The adventitious roots that develop after one-week transformation are cut and potato seedlings are transferred to ½ MS medium plates including salts plus 1% sucrose. Two-weeks after transformation, non-fully transformed potato roots are cut to allow the further growth of potato roots constitutively expressing AtPIN6. Fully transformed roots are inoculated, among others, with Sinorhizobium fredii NGR234 and IRGB74 strains.

### Example 10: Phenotypic Analysis of PIN6 Expression in Tomato

To test the impact of PIN6 overexpression in tomato (Solanum lycopersicum), tomato seeds (Solanum lycopersicum cv. Moneymaker) were sterilized first with 70% ethanol for three minutes and then with sodium hypochlorite (6%) plus Tween 20 (0.005%) for another twenty minutes. Subsequently, seeds were washed at least five times with sterile H₂O to remove any NLCO content. Seeds were allowed to germinate on ½ Murishage & Skoog (MS) medium including vitamins plus 3% sucrose. Six-week old tomato hairy roots (Solanum lycopersicum cv. Moneymaker*) were transformed with Agrobacterium Rhizogenes strain Arqua1 carrying a AtUBI10pro::AtPIN6-eGFP/SoylcACT2pro::NLS-2×mCherry construct. The inoculated tomato seedlings were placed on ½ MS medium plates including vitamins plus 3% sucrose, and the hypocotyl was covered with wet filter paper for one week. The adventitious roots that developed after one-week transformation were cut and tomato seedlings were transferred to ½ MS medium plates including salts plus 1% sucrose. Two-weeks after transformation, non-fully transformed tomato roots were cut to allow the further growth of tomato roots constitutively expressing AtPIN6. Fully transformed roots grown in soil+vermiculite were inoculated, among others, with IRBG-DsRed strain OD-0.4 (8.9×10\hat{ }6 CFU/mL) and harvested after 8 weeks for nodule quantification and imaging.

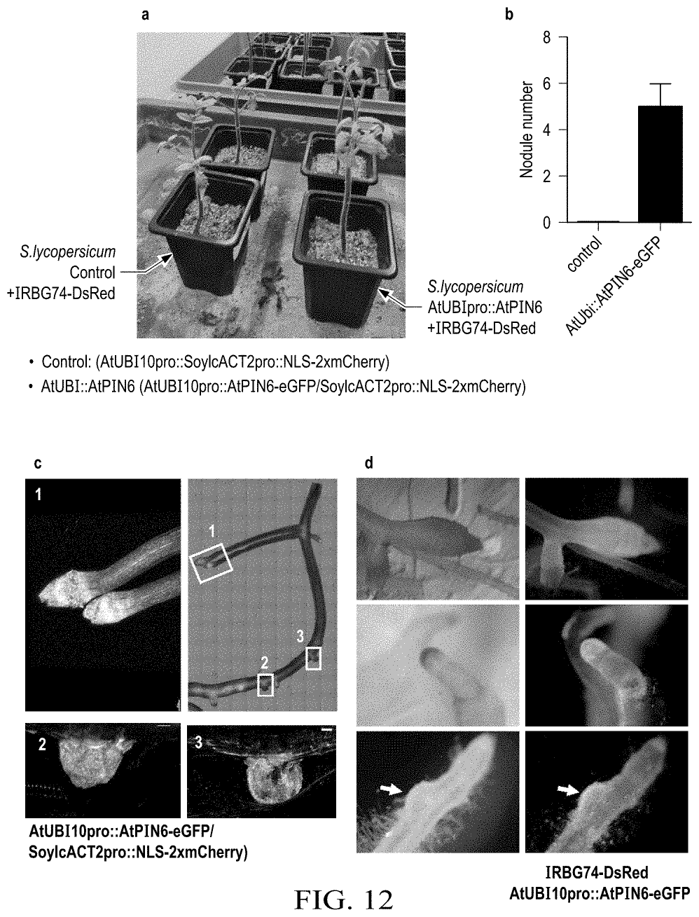

FIG. 12A shows transformed tomato plants grown in soil+vermiculite. FIG. 12B shows that PIN6-transformed plants form nodule-like structures while control plants do not. FIG. 12C, shows a root segment of a PIN6-transformed plant inoculated with IRBG74 DsRed. Numbers indicated boxed areas show nodule-like structures. FIG. 12D, left panel, shows bright field images of PIN6-transformed roots inoculated with IRBG74 DsRed, whereas the right panel shows PIN6-GFP and IRBG74 DsRed in these roots. The white arrow indicates a nodule structure.

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

What is claimed is:

1. A recombinant nucleic acid molecule comprising a heterologous promoter operably linked to a polynucleotide segment encoding an auxin transporter protein or fragment thereof, wherein:
a. said protein comprises the amino acid sequence of any of SEQ ID NOs: 3 and 5-14;
b. said protein comprises an amino acid sequence having at least 85%, or 90%, or 95%, or 98% or 99%, or about 100% amino acid sequence identity to any of SEQ ID NOs: 3 and 5-14;
c. said polynucleotide segment hybridizes under stringent hybridization conditions to a polynucleotide having the nucleotide sequence of any of SEQ ID NO: 1, 2, or 4; or
d. said polynucleotide segment comprises the nucleotide sequence of any of SEQ ID NO: 1, 2, or 4.

2. The recombinant nucleic acid molecule of claim 1, wherein said recombinant nucleic acid molecule is capable of producing an effective amount of the protein or fragment to form nodule-like structures in a plant in the presence of a Rhizobium bacterium when expressed in a plant cell.

3. The recombinant nucleic acid molecule of claim 1, wherein said recombinant nucleic acid molecule is capable of producing an effective amount of the protein or fragment to increase drought resistance as compared to a plant lacking said recombinant nucleic acid molecule when expressed in a plant cell.

4. The recombinant nucleic acid molecule of claim 1, wherein said recombinant nucleic acid molecule is in operable linkage with a vector.

5. The recombinant nucleic acid molecule of claim 4, wherein said vector is selected from the group consisting of a plasmid, phagemid, bacmid, cosmid, and a bacterial or yeast artificial chromosome.

6. The recombinant nucleic acid molecule of claim 1, further defined as comprised within a bacterial or plant host cell.

7. The recombinant nucleic acid molecule of claim 6, wherein said bacterial host cell is from a genus of bacteria selected from the group consisting of: Agrobacterium, Rhizobium, Bacillus, Brevibacillus, Escherichia, Pseudomonas, Klebsiella, Pantoea, and Erwinia.

8. The recombinant nucleic acid molecule of claim 7, wherein said Bacillus is Bacillus cereus or Bacillus thuringiensis, said Brevibacillus is a Brevibacillus laterosperous, and said Escherichia is a Escherichia coli.

9. The recombinant nucleic acid of claim 6, wherein said plant cell is a dicotyledonous or a monocotyledonous plant cell.

10. The recombinant nucleic acid of claim 9, wherein said plant cell is selected from the group consisting of an alfalfa, banana, barley, bean, broccoli, cabbage, brassica, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, watermelon, and wheat plant cell.

11. The recombinant nucleic acid molecule of claim 2, wherein expression of said protein produces the formation of nodule-like structures in a plant in the presence of a Rhizobium bacterium.

12. A plant or part thereof comprising the recombinant nucleic acid molecule of claim 1.

13. The plant of claim 12, wherein said plant is a non-legume plant.

14. The plant of claim 12, wherein said plant is a non-host plant.

15. The plant of claim 12, wherein said plant is a monocot plant or a dicot plant, or a part thereof.

16. The plant of claim 15, wherein the plant is selected from the group consisting of an alfalfa, banana, barley, bean, broccoli, cabbage, brassica, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, watermelon, and wheat plant.

17. The plant of claim 12, wherein the part of the plant thereof is a seed, and wherein said seed comprises said recombinant nucleic acid molecule.

18. A composition comprising the recombinant nucleic acid molecule of claim 1.

19. The composition of claim 18, further comprising a Rhizobium bacterium.

20. The composition of claim 19, wherein said bacterial species is selected from the group consisting of: Mesorhizobium loti, Sinorhizobium meliloti, Sinorhizobium fredii, Rhizobium sp. IRBG74 and NGR234, Bradyrhizobium sp., and Azorhizobium caulinodans ORS571.

21. A commodity product produced from the plant, or part thereof, of claim 10, wherein the commodity product comprises a detectable amount of said recombinant nucleic acid molecule, said protein, or a fragment thereof.

22. The commodity product of claim 21, selected from the group consisting of commodity corn bagged by a grain handler, corn flakes, corn cakes, corn flour, corn meal, corn syrup, corn oil, corn silage, corn starch, corn cereal, and the like, and corresponding soybean, rice, wheat, sorghum, pigeon pea, peanut, fruit, melon, and vegetable commodity products including, where applicable, juices, concentrates, jams, jellies, marmalades, and other edible forms of such commodity products containing a detectable amount of such polynucleotides and or polypeptides of this application, whole or processed cotton seed, cotton oil, lint, seeds and plant parts processed for feed or food, fiber, paper, biomasses, and fuel products such as fuel derived from cotton oil or pellets derived from cotton gin waste, whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts.

23. A method of producing progeny seed comprising crossing the plant of claim 12 with itself or a second plant and allowing seed to form.

24. A plant, wherein the cells of said plant comprise the recombinant nucleic acid molecule of claim 1.

25. A method for forming nodule-like structures in a plant, said method comprising expressing the recombinant nucleic acid molecule of claim 1 in said plant; and contacting said plant with an effective amount of one or more rhizobia bacteria.

26. The method of claim 25, wherein said nodule-like structures express the auxin transporter protein encoded by the recombinant nucleic acid molecule of claim 1.

27. The method of claim 26, further comprising selecting a plant comprising nodule-like structures.

28. A method of detecting the presence of the recombinant nucleic acid molecule of claim 1 in a sample comprising plant genomic DNA, comprising:
a. contacting said sample with a nucleic acid probe that hybridizes under stringent hybridization conditions with genomic DNA from a plant comprising said recombinant nucleic acid molecule, and does not hybridize under such hybridization conditions with genomic DNA from an otherwise isogenic plant that does not comprise said recombinant nucleic acid molecule, wherein said probe is homologous or complementary to SEQ ID NO: 1, 2, or 4; or a sequence that encodes a protein comprising an amino acid sequence having at least 85%, or 90%, or 95%, or 98% or 99%, or about 100% amino acid sequence identity to any of SEQ ID NOs: 3 or 5-14;
b. subjecting said sample and said probe to stringent hybridization conditions; and
c. detecting hybridization of said nucleic acid probe with said recombinant nucleic acid molecule.

29. A method of detecting the presence of a protein, or fragment thereof, in a sample comprising protein, wherein said protein comprises the amino acid sequence of any of SEQ ID NOs: 3 or 5-14; or said protein comprises an amino acid sequence having at least 85%, or 90%, or 95%, or 98% or 99%, or about 100% amino acid sequence identity to any of SEQ ID NOs: 3 or 5-14; comprising:
a. contacting said sample with an immunoreactive antibody; and
b. detecting the presence of said protein, or fragment thereof.

30. The method of claim 29, wherein the step of detecting comprises an ELISA, or a Western blot.


SEQUENCE LISTING (21 sequences)

SEQ ID NO: 1 - DNA, 3996 bp, Arabidopsis thaliana
  source 1..3996: mol_type = other DNA
atgataacgggaaacgaattctacacggtgatgtgtgccatggcaccactctacttcgccatgttcgtggcttatggatcggtaaagtggtgcaagatcttcacgccggcgcaatgttccggaatcaaccgttttgtctccgttttcgccgtcccagtcctctctttccacttcatctcacaaaacaacccttacaaaatggacacaatgttcatcctcgccgacactttgtccaaaatcttcgtcttcgttttgctctctctatgggccgttttcttcaaagccggtggtctcgattggctcatcactctattctcaatcgctactctccctaacactctcgttatgggcatacctttgcttcaggccatgtatggagattacactcaaaccctcatggtccaactcgttgttcttcaatgcatcatttggtacgttatcaaattactatccgttctttattcatttaccctgtgattgattagaaaacctcataataatggaattattttgtgatttcacatatttttaccacacattttttcatggattttgtaaccaaaacttaaatgaatttcgttactctaggatataattcaaccaaatcaaatgtttttactaacccaagcttcttattagttgaattatttaattataacgtatgataatataagctaagtgtatatacatgtataggtatactctgctactattcctctttgaactccgagcagcgaggttgcttatccgagcagagtttccgggtcaagcagccggatcaatcgccaagatccaagtcgacgatgacgtcatttccttagacggcatggatccgctccgtacagaaactgaaaccgatgttaacggtcggatcaggctcaggatccgacggtccgtatcatccgtacctgattctgttatgtcctcctccttgtgcttaacccctagagcctctaatctttctaacgccgagattttctccgtcaacactcctaataaccgtttcttccacggcggaggaggaagcggtacccttcagttttataacggtagtaacgagatcatgttttgtaacggagatttaggcgggttcggatttactcgacccggattaggtgcaagtcctagaaggctctcgggttatgcttcctccgatgcttactcgttgcagccaacaccacgtgcctcgaactttaacgagcttgacgttaacggaaacggtacgccggtttggatgaaatctcctgccgccgggagaatataccggcaatcttcgccgaagatgatgtgggagtctgggcaaagacatgcagccaaagatatcaatggtacgttttctttgaaagatgtgtcgttttcgtttttcgttagcactgacacgacggcgtttagtcaattttcttctcttttcgtgtttttctttatcattggttcttttaaaagatttaatgaatagaaaaacattgatagactacataaaatgcagcttttcagaaataaaaaaacttagcattgtctcttttactttccaaatccctaggaattaactaattagaaattcaatctaattttataaaatttataagtaatagtcactaaatcccacttattttatttttttgtgtggaaacttttgaattttgatacttcatgtttttgagcttaagattctataggacaacatgatgaagcatgtaaacaaagcggacccctcataccacccacacacttactatcatcaataaaatcaccaaaatggtcaacatgatgttgacttttttgtttctcatgatgtttttggactcatcaaattaatgtccattctaaagaatttatttgggagattctatcattttcctctagctcacgtgtgtaatcatcagcgaaaccgacgcatggttttctatttttttcttccattactaaatccacaacctttattaacaaaaagagttataactaattagttgctttcaaaatatcaagttttgtcctaactatcactctgtatctgctcacgtgtattttatataactaaggatcagtgccagagaaggagattagcttcagagacgcacttaaggctgcaccacaggctacggcagccggtggtggtgcttccatggaggaaggagccgccggaaaagacacaactccggtggctgcgattggcaagcaagaaatgccaagtgcaattgtaatgatgcgcctcatactaacagtcgtgggccgcaagctttctcgaaaccctaacacttattccagcctcttaggtcttgtctggtcacttatatccttcaagtgagtcaaaaaataactcaaatatcaaaatttcaactataatcattgaatttgtttgttatataactttaactcatattttattttgctaaataatgagcagatggaatataccgatgccaaatatagtggatttctcgataaagatcatttcagatgcaggtcttgggatggctatgtttagtttaggtatgaacatgttcttataaacctgcataataaataaaattatgtataacgtattctaacaaaattaaactataggcctgtttatggcgctgcagccaaagatgatcccttgtggggcgaaaaaggcaacaatggggatgttgatcaggttcatctcaggtcctcttttcatggctggtgcttcccttcttgtcggattaagaggttctaggttacatgctgctatcgtacaggtaaactattcagagtttaccttgatttggcccatatatattattgttggaaaattaaagaaaacatacttggtgatggagaaagtgtttaagattttgggagtgaattaggaagtttaacatatagaagggactaattagttaaaaaaaaatgaaggatagatatgttcatagctttcaaaagtacaaatatctatctcatataaacctaatatagtctaccattatatctagacttatatgttacaaagtatctagatattatgaaaatatctagatattttacatgatgcaattctcctgtattagtctaaggaggtattaatattttaagatggttctaaagagactctttaacttgtttcaagttttgcgtttggtccaagaaattaatgatacctggatcattgacacataattataactaatccaaaatcaagttaaatatttcaaaatataaattacccgtggctataatttaaacttcgtttcttataaaagttttcgattaacttatagtagagatatgtacatctaaatttgatgaagccgatatgttcttttaggctgctctaccgcaagggatagtaccatttgtgtttgcgagggagtacaatttgcatccagacctactcagtacattgtgagtctcttgctcttctttttgttgttcttcatttacatttacggttgaacgtttcaaaatgcggtattatggtttatgtgaaatctctgttcttggtgattttttgttttgttttcagggttatctttgggatgatagtatctttaccagtaactattttgtactacgtcctcttgggcctatgattcacacaacacactgaagaaaaagattacaccggagaaattaaatgatctatgaataaagaggtctggaaatagagaaatgtgagctcttaagagttgttcttgaggagcttgtgagaaagattaattaacggttaattacattgttgaatcaaattactggtaaatggttaggtgaataatttaattttatgtatttgatatccaacattgatcgacgtacgtacgtagtgaggtagaggcttaggttaggaatcaatcgtgaatactatttcaacgttgattcccgcgagctgaatattttttgtgatgtatagtaaaacgcatatgattataggaatcatcataaagattcttgtcatgaagaacaatattaaaccaaattatgcattacgagcaaaataattatacaaaaatactgataattataacttgggtcatt

SEQ ID NO: 2 - DNA, 1713 bp, Arabidopsis thaliana
  source 1..1713: mol_type = other DNA
atgataacgggaaacgaattctacacggtgatgtgtgccatggcaccactctacttcgccatgttcgtggcttatggatcggtaaagtggtgcaagatcttcacgccggcgcaatgttccggaatcaaccgttttgtctccgttttcgccgtcccagtcctctctttccacttcatctcacaaaacaacccttacaaaatggacacaatgttcatcctcgccgacactttgtccaaaatcttcgtcttcgttttgctctctctatgggccgttttcttcaaagccggtggtctcgattggctcatcactctattctcaatcgctactctccctaacactctcgttatgggcatacctttgcttcaggccatgtatggagattacactcaaaccctcatggtccaactcgttgttcttcaatgcatcatttggtatactctgctactattcctctttgaactccgagcagcgaggttgcttatccgagcagagtttccgggtcaagcagccggatcaatcgccaagatccaagtcgacgatgacgtcatttccttagacggcatggatccgctccgtacagaaactgaaaccgatgttaacggtcggatcaggctcaggatccgacggtccgtatcatccgtacctgattctgttatgtcctcctccttgtgcttaacccctagagcctctaatctttctaacgccgagattttctccgtcaacactcctaataaccgtttcttccacggcggaggaggaagcggtacccttcagttttataacggtagtaacgagatcatgttttgtaacggagatttaggcgggttcggatttactcgacccggattaggtgcaagtcctagaaggctctcgggttatgcttcctccgatgcttactcgttgcagccaacaccacgtgcctcgaactttaacgagcttgacgttaacggaaacggtacgccggtttggatgaaatctcctgccgccgggagaatataccggcaatcttcgccgaagatgatgtgggagtctgggcaaagacatgcagccaaagatatcaatggatcagtgccagagaaggagattagcttcagagacgcacttaaggctgcaccacaggctacggcagccggtggtggtgcttccatggaggaaggagccgccggaaaagacacaactccggtggctgcgattggcaagcaagaaatgccaagtgcaattgtaatgatgcgcctcatactaacagtcgtgggccgcaagctttctcgaaaccctaacacttattccagcctcttaggtcttgtctggtcacttatatccttcaaatggaatataccgatgccaaatatagtggatttctcgataaagatcatttcagatgcaggtcttgggatggctatgtttagtttaggcctgtttatggcgctgcagccaaagatgatcccttgtggggcgaaaaaggcaacaatggggatgttgatcaggttcatctcaggtcctcttttcatggctggtgcttcccttcttgtcggattaagaggttctaggttacatgctgctatcgtacaggctgctctaccgcaagggatagtaccatttgtgtttgcgagggagtacaatttgcatccagacctactcagtacattggttatctttgggatgatagtatctttaccagtaactattttgtactacgtcctcttgggcctatga

SEQ ID NO: 3 - protein, 570 aa, Arabidopsis thaliana
MITGNEFYTVMCAMAPLYFAMFVAYGSVKWCKIFTPAQCSGINRFVSVFAVPVLSFHFISQNNPYKMDTMFILADTLSKIFVFVLLSLWAVFFKAGGLDWLITLFSIATLPNTLVMGIPLLQAMYGDYTQTLMVQLVVLQCIIWYTLLLFLFELRAARLLIRAEFPGQAAGSIAKIQVDDDVISLDGMDPLRTETETDVNGRIRLRIRRSVSSVPDSVMSSSLCLTPRASNLSNAEIFSVNTPNNRFFHGGGGSGTLQFYNGSNEIMFCNGDLGGFGFTRPGLGASPRRLSGYASSDAYSLQPTPRASNFNELDVNGNGTPVWMKSPAAGRIYRQSSPKMMWESGQRHAAKDINGSVPEKEISFRDALKAAPQATAAGGGASMEEGAAGKDTTPVAAIGKQEMPSAIVMMRLILTVVGRKLSRNPNTYSSLLGLVWSLISFKWNIPMPNIVDFSIKIISDAGLGMAMFSLGLFMALQPKMIPCGAKKATMGMLIRFISGPLFMAGASLLVGLRGSRLHAAIVQAALPQGIVPFVFAREYNLHPDLLSTLVIFGMIVSLPVTILYYVLLGL

SEQ ID NO: 4 - DNA, 1668 bp, Lotus Japonicus
  source 1..1668: mol_type = other DNA
atggggattcctctactccaagccatgtacggccaattcacgcaaggcctcatggtgcaactcgttgtactccaatgcatcatatggtatactctactgttgtttctattggaatacagagcagcgacccttttgatcaaaaccgagttccctggggacacggcggcgtccataaccaaaatcgaggtcgacggcgacgtgatttccctcgacggccacgacgtaccactgcaaacgcagtcggaaaccgacacccacggccgcatcagcgtgcgaatccggtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcgatcaatctcctcagctccggactcaacctcatcaatcggcaacgccgttatcactccaaggcattcgaatctcaccaacgccgagcacctgctcgacggcgacccctcctttggatacccgccggcgagcccgaggttatccggatgcgcctcctccgacgcgtactcgcttcagcccacgccgcgggcgtcaaatttcaatgagacggaggtcacggcgggaacgccggtatgggggaggtctacggtgggcggcgggagggtttctcggcagatgtcgccggggaaatgccaagtagaagagagacagggatgcaaggacattacgcagtcagataaagaaattagcttcagagacaacatcaaagtttcaatgccaggggaagaagctgcagacacaactgctcgtaaccagaaaatgccacatgcttttgtcatgataaggcttatacttacagtagtgggaagaaaactttcgcgtaaccctaatacatattctagtgtattaggacttgtttggtctataatctccttcaaatggaacatggaaatgcctagtctgatcaaagcatctatcaaaatcatctcggatgcaagccttggaatggctatgtttagcttagggcttttcatggcccttcagcctcgtatcattgcttgtggtaccaaaagagcagccatgggaatggccattcgtttcgtgtgcgggcctcttgtaatgtcagcatcctccattgtcattggattgagaggggacaaattgcacacagcaattgtacaggctgcacttccacaggggattgtaccatttgtatttgcaagggaatatgggctgcatcctgatattttgagcacaggggttatctttggcatgctagtatccttaccaataactctcctatattacatacttcttggcttgtga

SEQ ID NO: 5 - protein, 517 aa, Lotus Japonicus
MVTGDDLYNVMCAMVPLYFAMLVAYASVKWCKMFTPQQCSGVNRFVAVFAIPVLSFHFISINNPYEMDAKFIIADTLSKLVVLFFLFLWAIFFASGSLDWLITLFSLATLPNTLVMGIPLLQAMYGQFTQGLMVQLVVLQCIIWYTLLLFLLEYRAATLLIKTEFPGDTAASITKIEVDGDVISLDGHDVPLQTQSETDTHGRISVRIRRSISSAPDSTSSIGNAVITPRHSNLTNAEHLLDGDPSFGYPPASPRLSGCASSDAYSLQPTPRASNFNETEVTAGTPVWGRSPVGGGRVSRQMSPGKCQVEERQGCKDITQSDKEISFRDNIKVSMPGEEAADTTARNQKMPHAFVMIRLILTVVGRKLSRNPNTYSSVLGLVWSLISFKWNMEMPSLIKASIKIISDASLGMAMFSLGLFMALQPRIIACGTKRAAMGMAIRFVCGPLVMSASSIVIGLRGDKLHTAIVQAALPQGIVPFVFAREYGLHPDILSTGVIFGMLVSLPITLLYYILLGL

SEQ ID NO: 6 - protein, 362 aa, Brachypodium distachyon
MISWIAIYHVLEATVPLYVAMILAYLSIKWWKLFTPEQCSGINKFVAKFSIPLLSFQVISTNNPYDMNLKLILADILQKLLALLGFAAISRACCAEKFDWLITGFSLSTLPNTLIVGIPLLKGMYGDEALNLISQIVVLQSLIWYTLLLFLLEFRAAKGIVPTTSSEITAEEEPGTLGTTQQRYQEGQAKGVSAKCSSAFNFLLVVGKKLVMNPNMYACLIGLIWALVSFRWQIQLPVIISNSIRILSDGGLGMAMFSLGLFTALQTKIIVCGTKRMLLSLGIRFFLGPALMVISSYAVGMRGILLKVAIVQAALPQGIVPFVFAKEYNLHADILSTAIIVGMMVAVPAALAYYFVLDQPRF

SEQ ID NO: 7 - protein, 507 aa, Musa acuminata
MITWEDFYTVMCAMVPLYFAMFVAYGSVKWWKIFTPEQCSGINRFVATFAVPVLSFHFISHNNPYQMDSRFILADTLSKLLVLVILSLWASLSAACAGPRRRGRRLDWVITLFSVGTLPNTLVMGIPLLRAMYGDFTQSLMVQLVVLQCIIWYTLLLFLFEYRAATMLIEDQFPGTAAAAIAKFEVDGDIISLDGRDPIRAESEMEIGTPVWVRSPAAPAGMLIRQQSPATPGALKLASPLWTLLAIGSRRSQSSLHQFARIKLHHLRESRSHSPFILLGMGLEAVKETCVTTATVPVLHITDTRLEGREKDLSFRSTSKFVVDKEDEEEELEVEGDQEMPAAFVMLRLILTVLGRKLSRNPNTYSSILGLLWSLISFKWGVDMPTLVKESIKIISDAGLGMAMFSLGLFMALQPRIIACGPKMAAISMAIRFLSGPMVMSVASMVVGLRGVRLHTAIVQAALPQGIVPFVFAREYGLHPDILSTGVIFGMLVSLPVTLLYYILLGL

SEQ ID NO: 8 - protein, 572 aa, Musa acuminata
MITWEDFYTVMCAMIPLYFAMFLAYGSVKWWKIFTPEQCSGINRFVATFAVPVLSFHFISHNNPYQMDSRFILADTLSKLLVLMAISLWAALSAAFASPRRRGRRLDWVITLFSVGTLPNTLVMGIPLLRAMYGDFTQSLMVQLVVLQCIIWYTLLLFLFEYRAATLLIEDQFPGTAAAAIAKFEIDGDIISLDGRDPIQAESEIDADGRLRVRIRRSTSSAPGSGMSSSLGITPRRLPHPSGYASSDAYSLQPTPRASNFNELEICTPVWSTRMSGVSTHMPSAAVRSLFGLCLPWHHIEDKEKTSHYSDTAVIYNTTQIHGLRFFNNVSFLEQHCCYSHVMAGSSAAVTVQGCRRCSLSLRMPCNLESHYVLGEKDLSFRSTSKFVVRKEDEIEMEGEAEQEMPAALVMLRLILTVVGRKLSRNPNTYSSILGLLWSLISFKWDISMPSLLKESIKIISDAGLGMAMFSLGLFMALQPRIIACGPKMAAISMAIRFLSGPTVMSAASMAVGLRGVRLHTAIVQAALPQGIVPFVFAREYGLHPDILSTGVIFGMLVSLPVTLLYYILLGL

SEQ ID NO: 9 - protein, 527 aa, Vitis vinifera
MISADDFYKVMCAMVPLYFAMLVAYGSVKWCKIFSPEQCSGINRFVAVFAVPVLSFHFISQNNPYEMDTKFIVADTLSKLLVLVLLSVWAILFKGGLDWLITLFSLATLPNTLVMGIPLLNAMYGDFTQSLMVQLVVLQCIIWYTLLLFLFEYRAATLLIKNQFPGSTAASISKFEIDGDVISLDGRDPVRTESEIDGNGRIRVRIRRSTSSAPDSALSSSMGITPRASNLSGAEIFSVNTPAPLHDPRLSGYASSDAYSLQPTPRASNFNELDTTTITTTNTPFWVRSPVAGKVYRQQSPAFSGVRMVWESPGKCNGGGGERQGCKDVVGEREISFRDSSKFPVAEEEDPKATATGQDMPRTHVMLRLILTMVGRKLSRNPNTYSSVLGLLWSLISFKWNVGMPSLVKYSIKIISDAGLGMAMFSLGLFMALQPRIIACGTKRATMGMGIRFICGPILMSAASIAVGLRGVRLHAAIVQASLPQGIVPFVFAREYGLHPDILSTGVIFGMLVSLPVTLLYYILLGL

SEQ ID NO: 10 - protein, 520 aa, Solanum lycopersicum
MIGVNDFYKVMCAMVPLYFAMIVAYGSVKWWKIFSPEQCSGINRFVAVFAVPVLSFHFISQNNPYQMDTKFILADTLSKILVLVLLSVWAICKGQLDWLITLFSVSTLPNTLVMGIPLLNAMYGDFTQSLMVQLVVLQCIIWYTLLLFLFEYRAATILIKNQFPGNVAASITKFEIDNDVISLDGRNPLCTESEIDGNGRIHVRIRRSTSSAPESAFSSSIGITPRASNLSNAEIFSVHTPLHNGDIPFGHGDLGVGFRAASPRLSGGYASSDAYSLQPTPRASNFNELDVTTSGNTPMWVMSPVGGKVFKQASPSSKMAWESSCLNGERQGYRDDVGEKEISFRDISNFPVQEVGAADSSNTNNIIKQEMPNALVMLRLIIVMVGRKLSRNPNTYSSILGLLWSLISFKWNVGMPSLVKYSIKIISDAGLGMAMFSLAIGMAIRFIGGPLVMSATSIAVGLKGVRLHTAIVQAALPQGIVPFVFAREYGLHPDILSTGVIFGMLVSLPVTLLYYVLLGL

SEQ ID NO: 11 - protein, 552 aa, Populus trichocarpa
MITADDFYKVMCAMVPLYFAMLVAYGSVKWYKIFTPEQCSGINRFVAVFAVPVLSFHFIAQNNPYQMDTKFILADTLSKVLALVLLSVWAVFFNGEFDWLITLFSVATLPNTLVMGIPLLKAMYGDFTQSLMVQVVVLQCIIWYTLLLFLFEYRAATLLIKAQFPGPTAATISKIELDDDVISLDGRDPLRTESETDGNGRIRVRIRRSTSSAPDSALSSSICLTPRPSNLSNAEVFSVNTPVPLHEYHGYNGHFSHGPNNEIVLCNGDLGLAYRSGTSPRLSGYASSDAYSLQPTPRASNFNELDLTNATNTPFWVRSPVAGKIYRQPSPAVPEVKLVWGESPVICPNGGGKDVPGKEISFRDSCKMPAPEESNSKESVSNQEMPHAIVMIRLILVVVGRKLSRNPNTYSSVLGLLWSLASFKWNVGMPSLVKYSIKIISDAGLGMAMFSLGLFMALQPRIIVCGKKRATMAMAIRFICGPIVMSTTSVAVGMRGVRLRAAIVQAALSQGIVPFVFAREYGLHPDIMSTGVIFGMLVSLPVTLIYYIFLGL

SEQ ID NO: 12 - protein, 553 aa, Populus trichocarpa
MITAGDFYKVMCAMVPLYFAMLVAYGSVKRYKIFTPEQCSGINRFVAVFAVPVLSFHFIAQNNPYQMDTRFIMADTLSKVLVLVLLSIWVIFFNGGLDWLITLFSIATLPNTLVMGIPLLKAMYGDFTQSLMVQVVVLQCIIWYTLLLFLFEYRAATLLIKTQFPGPKAASISKIELDNDVISLDGRDPLRTESETDGNGRLRVRIRRSTSSAPDSALSSSICLTPRPSNLSNAEVFSVSTPAPLQEYHGYNGRFSHGPNNEIMLCNGDLGFGYHRSGTSPRLSGYASSDAYSLQPTPRTSNFNEWDLTNATNTPFWARSPVAGKISRHPSPAVPELRLVWGESPVMCPNGGGKEIAEKEISFRDSCKMPAPEETNSKEAVTSQEMPHAIVMLRLILLVVGRKLSRNPNTYSSVLGLLWSLVSFKWNVGMPSLVKYSIKIISDAGLGMAMFSLGLFMALQPRIIVCGKKRATMAMAIRFICGPVVMSTASLAVGMRGERLRAAIVQAALSQGIVPFVFAREYGLHPDIMSTGVIFGMLVSLPVTLLYYIFIGL

SEQ ID NO: 13 - protein, 311 aa, Oryza sativa
MVSWKDIYLVLEATVPLYVAMILAYLSIKWWKLFTPEQCSGINKFVAKFSIPLLSFQVISTTDPYDMNIKLIYSDILQKSLALLGFAAISKACCAEKFDWLITGFSLSTLPNTLIVGIPLLKGMYGEQAGKLLSQIVVLQSLIWYTLLLFLFELRAANGMATTTSSETTGLIWALVGFRWHIRLPLIVSNSIRMLSDGGLGMAMFSLGLFTALQTKIIACGAKRMLLALAIRFFLGPALMGMSSYAIGMRGVLLKIAIVQAALPQGIVPFVFAKEYNVQADILSTAIIVGMMVAVPVALAYYFAMIIPAIK

SEQ ID NO: 14 - protein, 362 aa, Zea mays
MISWPTIYHVLEETVPLYVAMIVAYLSIQWWKLFTPEQCSGINKFVAKFSIPLLSFQILSTNNPYSMNLKLIFSDVLQKSLSLLGFAVASRVCCEEKFDWLITGFSLSTLPNTLIVGIPLLKGMYGDEAVKLLSQIVALQSLIWYTLLLFLFEFRAARGMAITTSSEAIDEAESGTPGPMQQGHEEGQAKGVSARCYGAFHFLLVVSRKLVTNPNMYASLAGLIWALISFRWRIQLPSIVNNSIRILSDGGLGMAMFSLGVFTALQTKIVACGTKRMLLSLGIRFFLGPGLMVISSYLIGMRGTLLKVAIIQAALPQGIVPFVFAKEYNVHADILSTAIILGMMVAVPVALGYYFVMDHPRL

SEQ ID NO: 15 - protein, 622 aa, Arabidopsis thaliana
MITAADFYHVMTAMVPLYVAMILAYGSVKWWKIFTPDQCSGINRFVALFAVPLLSFHFIAANNPYAMNLRFLAADSLQKVIVLSLLFLWCKLSRNGSLDWTITLFSLSTLPNTLVMGIPLLKGMYGNFSGDLMVQIVVLQCIIWYTLMLFLFEYRGAKLLISEQFPDTAGSIVSIHVDSDIMSLDGRQPLETEAEIKEDGKLHVTVRRSNASRSDIYSRRSQGLSATPRPSNLTNAEIYSLQSSRNPTPRGSSFNHTDFYSMMASGGGRNSNFGPGEAVFGSKGPTPRPSNYEEDGGPAKPTAAGTAAGAGRFHYQSGGSGGGGGAHYPAPNPGMFSPNTGGGGGTAAKGNAPVVGGKRQDGNGRDLHMFVWSSSASPVSDVFGGGGGNHHADYSTATNDHQKDVKISVPQGNSNDNQYVEREEFSFGNKDDDSKVLATDGGNNISNKTTQAKVMPPTSVMTRLILIMVWRKLIRNPNSYSSLFGITWSLISFKWNIEMPALIAKSISILSDAGLGMAMFSLGLFMALNPRIIACGNRRAAFAAAMRFVVGPAVMLVASYAVGLRGVLLHVAIIQAALPQGIVPFVFAKEYNVHPDILSTAVIFGMLIALPITLLYYILLGL

SEQ ID NO: 16 - protein, 647 aa, Arabidopsis thaliana
MITGKDMYDVLAAMVPLYVAMILAYGSVRWWGIFTPDQCSGINRFVAVFAVPLLSFHFISSNDPYAMNYHFLAADSLQKVVILAALFLWQAFSRRGSLEWMITLFSLSTLPNTLVMGIPLLRAMYGDFSGNLMVQIVVLQSIIWYTLMLFLFEFRGAKLLISEQFPETAGSITSFRVDSDVISLNGREPLQTDAEIGDDGKLHVVVRRSSAASSMISSFNKSHGGGLNSSMITPRASNLTGVEIYSVQSSREPTPRASSFNQTDFYAMFNASKAPSPRHGYTNSYGGAGAGPGGDVYSLQSSKGVTPRTSNFDEEVMKTAKKAGRGGRSMSGELYNNNSVPSYPPPNPMFTGSTSGASGVKKKESGGGGSGGGVGVGGQNKEMNMFVWSSSASPVSEANAKNAMTRGSSTDVSTDPKVSIPPHDNLATKAMQNLIENMSPGRKGHVEMDQDGNNGGKSPYMGKKGSDVEDGGPGPRKQQMPPASVMTRLILIMVWRKLIRNPNTYSSLFGLAWSLVSFKWNIKMPTIMSGSISILSDAGLGMAMFSLGLFMALQPKIIACGKSVAGFAMAVRFLTGPAVIAATSIAIGIRGDLLHIAIVQAALPQGIVPFVFAKEYNVHPDILSTAVIFGMLVALPVTVLYYVLLGL

SEQ ID NO: 17 - protein, 640 aa, Arabidopsis thaliana
MISWHDLYTVLTAVIPLYVAMILAYGSVRWWKIFSPDQCSGINRFVAIFAVPLLSFHFISTNNPYAMNLRFIAADTLQKIIMLSLLVLWANFTRSGSLEWSITIFSLSTLPNTLVMGIPLLIAMYGEYSGSLMVQIVVLQCIIWYTLLLFLFEFRGAKMLIMEQFPETAASIVSFKVESDVVSLDGHDFLETDAEIGDDGKLHVTVRKSNASRRSFCGPNMTPRPSNLTGAEIYSLSTTPRGSNFNHSDFYNMMGFPGGRLSNFGPADMYSVQSSRGPTPRPSNFEENCAMASSPRFGYYPGGGAGSYPAPNPEFSSTTTSTANKSVNKNPKDVNTNQQTTLPTGGKSNSHDAKELHMFVWSSNGSPVSDRAGLNVFGGAPDNDQGGRSDQGAKEIRMLVPDQSHNGETKAVAHPASGDFGGEQQFSFAGKEEEAERPKDAENGLNKLAPNSTAALQSKTGLGGAEASQRKNMPPASVMTRLILIMVWRKLIRNPNTYSSLIGLIWALVAFRWHVAMPKIIQQSISILSDAGLGMAMFSLGLFMALQPKLIACGNSVATFAMAVRFLTGPAVMAVAAIAIGLRGDLLRVAIVQAALPQGIVPFVFAKEYNVHPAILSTGVIFGMLIALPITLVYYILLGL

SEQ ID NO: 18 - protein, 612 aa, Arabidopsis thaliana
MITWHDLYTVLTAVVPLYVAMILAYGSVQWWKIFSPDQCSGINRFVAIFAVPLLSFHFISTNDPYAMNFRFVAADTLQKIIMLVLLALWANLTKNGSLEWMITIFSLSTLPNTLVMGIPLLIAMYGTYAGSLMVQVVVLQCIIWYTLLLFLFEYRGAKLLIMEQFPETGASIVSFKVESDVVSLDGHDFLETDAEIGNDGKLHVTVRKSNASRRSLMMTPRPSNLTGAEIYSLSSTPRGSNFNHSDFYSVMGFPGGRLSNFGPADLYSVQSSRGPTPRPSNFEENNAVKYGFYNNTNSSVPAAGSYPAPNPEFSTGTGVSTKPNKIPKENQQQLQEKDSKASHDAKELHMFVWSSSASPVSDVFGGGAGDNVATEQSEQGAKEIRMVVSDQPRKSGGDDIGGLDSGEGEREIEKATAGLNKMGSNSTAELEAAGGDGGGNNGTHMPPTSVMTRLILIMVWRKLIRNPNTYSSLIGLIWALVAYRWHVAMPKILQQSISILSDAGLGMAMFSLGLFMALQPKIIACGNSVATFAMAVRFITGPAIMAVAGIAIGLHGDLLRIAIVQAALPQGIVPFVFAKEYNVHPTILSTGVIFGMLIALPITLVYYILLGL

SEQ ID NO: 19 - protein, 351 aa, Arabidopsis thaliana
MINCGDVYKVIEAMVPLYVALILGYGSVKWWHIFTRDQCDAINRLVCYFTLPLFTIEFTAHVDPFNMNYRFIAADVLSKVIIVTVLALWAKYSNKGSYCWSITSFSLCTLTNSLVVGVPLAKAMYGQQAVDLVVQSSVFQAIVWLTLLLFVLEFRKAGFSSNNISDVQVDNINIESGKRETVVVGEKSFLEVMSLVWLKLATNPNCYSCILGIAWAFISNRWHLELPGILEGSILIMSKAGTGTAMFNMGIFMALQEKLIVCGTSLTVMGMVLKFIAGPAAMAIGSIVLGLHGDVLRVAIIQAALPQSITSFIFAKEYGLHADVLSTAVIFGMLVSLPVLVAYYAALEFIH

SEQ ID NO: 20 - protein, 619 aa, Arabidopsis thaliana
MITWHDLYTVLTAVIPLYVAMILAYGSVRWWKIFSPDQCSGINRFVAIFAVPLLSFHFISSNNPYAMNLRFIAADTLQKLIMLTLLIIWANFTRSGSLEWSITIFSLSTLPNTLVMGIPLLIAMYGEYSGSLMVQIVVLQCIIWYTLLLFLFEYRGAKILIMEQFPETGASIVSFKVESDVVSLDGHDFLETDAQIGDDGKLHVTVRKSNASRRSFYGGGGTNMTPRPSNLTGAEIYSLNTTPRGSNFNHSDFYSMMGFPGGRLSNFGPADMYSVQSSRGPTPRPSNFEESCAMASSPRFGYYPGGAPGSYPAPNPEFSTGNKTGSKAPKENHHHVGKSNSNDAKELHMFVWGSNGSPVSDRAGLQVDNGANEQVGKSDQGGAKEIRMLISDHTQNGENKAGPMNGDYGGEEESERVKEVPNGLHKLRCNSTAELNPKEAIETGETVPVKHMPPASVMTRLILIMVWRKLIRNPNTYSSLIGLIWALVAFRWDVAMPKIIQQSISILSDAGLGMAMFSLGLFMALQPKLIACGNSTATFAMAVRFFTGPAVMAVAAMAIGLRGDLLRVAIVQAALPQGIVPFVFAKEYNVHPAILSTGVIFGMLIALPITLVYYILLGL

SEQ ID NO: 21 - protein, 367 aa, Arabidopsis thaliana
MISWLDIYHVVSATVPLYVSMTLGFLSARHLKLFSPEQCAGINKFVAKFSIPLLSFQIISENNPFKMSPKLILSDILQKFLVVVVLAMVLRFWHPTGGRGGKLGWVITGLSISVLPNTLILGMPILSAIYGDEAASILEQIVVLQSLIWYTILLFLFELNAARALPSSGASLEHTGNDQEEANIEDEPKEEEDEEEVAIVRTRSVGTMKILLKAWRKLIINPNTYATLIGIIWATLHFRLGWNLPEMIDKSIHLLSDGGLGMAMFSLGLFMASQSSIIACGTKMAIITMLLKFVLGPALMIASAYCIRLKSTLFKVAILQAALPQGVVPFVFAKEYNLHPEIISTGVIFGMLIALPTTLAYYFLLDL